(19) United States
(12) Patent Application Publication
(10) Pub. No.: US 2026/0047804 A1
(43) Pub. Date: Feb. 19, 2026

(54) REAL-TIME SURGICAL IMAGING WITH ENERGY TRANSMISSIVE BED TOP

(71) Applicant: California Institute of Technology, Pasadena, CA (US)

(72) Inventor: Lihong Wang, Arcadia, CA (US)

(73) Assignee: California Institute of Technology, Pasadena, CA (US)

(21) Appl. No.: 19/297,863

(22) Filed: Aug. 12, 2025

Related U.S. Application Data
(60) Provisional application No. 63/682,464, filed on Aug. 13, 2024.

(51) Int. Cl. A61B 5/00; A61B 8/00

(52) U.S. Cl. CPC A61B 5/704; A61B 5/0035; A61B 5/0093; A61B 5/7425; A61B 8/4281; A61B 8/4483; A61B 8/463; A61B 8/5261

(57) ABSTRACT

Real-time surgical imaging techniques with an energy transmissive surgical bed top having energy delivery/receiving systems above and below the bed top surface.

### CROSS-REFERENCE TO RELATED APPLICATION

This application claims benefit of and priority to U.S. Provisional Patent Application No. 63/682,464, titled “Comprehensive Real-Time Imaging Surgical Platform (CRISP),” and filed on Aug. 13, 2024, which is incorporated by reference herein in its entirety and for all purposes.

### FIELD

Certain aspects relate generally to medical imaging, and more specifically, to surgical imaging.

### BACKGROUND

Currently 20-25% of women undergoing lumpectomy surgery require additional surgeries. These surgeries may be needed to remove cancerous tissue that was not removed during the initial lumpectomy. Accurate demarking of tumors through imaging can help ensure complete removal of cancerous tissue and preserve healthy tissue.

Background and contextual descriptions contained herein are provided solely for the purpose of generally presenting the context of the disclosure. Much of this disclosure presents work of the inventor, and simply because such work is described in the background section or presented as context elsewhere herein does not mean that such work is admitted prior art.

### SUMMARY

Certain techniques disclosed herein may be practiced with a processor-implemented method, a system comprising one or more processors and one or more processor-readable media, and/or one or more non-transitory processor-readable media.

Certain embodiments pertain to real-time surgical imaging systems. In various implementations, a real-time surgical imaging system comprises a bed top surface configured to receive at least a portion of a patient, an energy transmissive layer, and one or more first energy delivery systems configured to deliver energy from below the bed top surface through the energy transmissive layer to a region of interest in the patient, and a detection system for detecting energy received from the region of interest.

Certain embodiments pertain to energy transmissive surgical bed top apparatus. In various implementations, an energy transmissive surgical bed top apparatus comprises a fluid container configured to acoustically couple to at least a portion of a patient being imaged, an energy transmissive transparent layer, a tank with a coupling medium, wherein the energy transmissive layer is located between the fluid container and the tank, an ultrasonic transducer array in the tank, the ultrasonic transducer array configured to detect energy received through the energy transmissive layer, and a mechanism coupled to the ultrasonic transducer array to rotate and/or translate the ultrasonic transducer array during operation.

Certain embodiments pertain to real-time surgical imaging method. In various implementations, a real-time surgical imaging method comprises acquiring first energy signals associated with energy delivered during a surgical operation by one or more first energy delivery systems to a region of interest of a patient from below a bed top surface through an energy transmissive layer, reconstructing one or more first inoperative images of the region of interest based on the energy signals, co-registering the one or more first images with one or more corresponding pre-operative images, displaying co-registered images.

These and other features and embodiments will be described in more detail with reference to the drawings.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### BRIEF DESCRIPTION OF THE DRAWINGS

FIG. 1 is a block diagram of components of a real-time surgical imaging system with an energy transmissive surgical bed top apparatus, according to certain implementations.

FIG. 2 is a schematic drawing of an example of components of a real-time surgical imaging system including an energy transmissive surgical bed top apparatus, according to various implementations.

FIG. 3 is a schematic drawing of an example of components of a real-time surgical imaging system including an energy transmissive surgical bed top apparatus, according to various implementations.

FIG. 4A is a drawing of a side view of components of a real-time surgical imaging system with an example of an energy transmissive surgical bed top apparatus configured to couple to and/or decouple from an existing surgical bed top, according to implementations.

FIG. 4B is a drawing of a side view of components of a real-time surgical imaging system with an example of an energy transmissive surgical bed top apparatus configured to couple to a standard support assembly, according to implementations.

FIG. 5A is a drawing of an isometric view of components of an example of an energy transmissive surgical bed top apparatus, according to certain implementations.

FIG. 5B is a cross-sectional view of the energy transmissive surgical bed top apparatus in FIG. 5A.

FIG. 6 is a drawing of an isometric view of a cutaway portion of components of an example of an energy transmissive surgical bed top apparatus, according to certain implementations.

FIG. 7A is a drawing of an isometric view of components of an example of an energy transmissive surgical bed top apparatus, according to certain implementations.

FIG. 7B is a cross-sectional view of the energy transmissive surgical bed top apparatus in FIG. 7A.

FIG. 8 is a drawing depicting a real-time surgical imaging system including an energy transmissive surgical bed top apparatus with energy delivery systems in a first position, according to various implementations.

FIG. 9 is a drawing depicting the real-time surgical imaging system in FIG. 8 with energy delivery systems in a second position.

FIG. 10 is a drawing depicting a real-time surgical imaging system with a robotic system, according to an embodiment.

FIG. 11 depicts a flowchart of a real-time surgical imaging method, according to embodiments.

FIG. 12 depicts a flowchart of a real-time surgical imaging method, according to embodiments.

FIG. 13 depicts a block diagram of an example of a computing device, according to various embodiments.

FIG. 14 depicts a block diagram of an example of a computing device, according to various embodiments.

The figures and components therein may not be drawn to scale.

### DETAILED DESCRIPTION

Different aspects are described below with reference to the accompanying drawings. In the following description, numerous specific details are set forth in order to provide a thorough understanding of the presented embodiments. The disclosed embodiments may be practiced without one or more of these specific details. In other instances, well-known operations have not been described in detail to avoid unnecessarily obscuring the disclosed embodiments. While the disclosed embodiments will be described in conjunction with the specific embodiments, it will be understood that it is not intended to limit the disclosed embodiments.

### I. Introduction

Disclosed herein are real-time surgical imaging techniques that employ an energy transmissive surgical bed top apparatus and one or more energy delivery systems. The energy delivery systems can provide energy to a region of interest of a patient disposed on the energy transmissive bed top apparatus of an operating table. For example, energy delivery systems may deliver energy from beneath the bed top surface through the energy transmissive bed top apparatus, from the side of the bed top, and/or from above the surgical field. The energy delivery systems may be free-space energy delivery systems and/or guided energy-delivery systems. A detection system measures energy received from the region of interest to generate energy signals. For example, laser light energy may be delivered to the region of interest by optical fibers and the light energy may induce photoacoustic waves which are measured by an acoustic transducer array to generate acoustic signals. Image data from the detected energy signals is processed to generate one or more surgical images (e.g., 2D images, 3D images, or both). The surgical images may be co-registered with the pre-operative images to provide guidance during surgeries which can reduce errors and improve outcomes. With these real-time surgical imaging techniques, surgical images may be acquired without having to move the patient from the transmissive bed top apparatus which can reduce errors and potential risks. As used herein, “real-time imaging” generally refers to image reconstruction within milliseconds (e.g., 30 milliseconds) of receiving the detected energy signals to enable providing visualizations back to a user of the surgical imaging system.

Traditional surgical procedures often require multiple devices for imaging and energy delivery, which can lead to workflow inefficiencies and potential risks. For example, a patient may need to be moved to a room near the operating room with a magnetic resonance imaging (MRI) system to take MRI images for guidance during surgery and moved back to the operating room. Certain examples of real-time surgical imaging systems discussed herein provide a single surgical platform that can implement multi-modal imaging by employing integrated energy delivery systems including free-space energy delivery systems and/or guided energy delivery systems. This surgical platform can provide real-time, surgical imaging guidance that combines surgically compatible imaging modalities with multiple energy delivery systems to enhance surgical precision and outcomes. Integrating multiple imaging modalities within a single platform that supports real-time imaging during surgeries can significantly enhance surgical capabilities. The real-time surgical imaging techniques may also align pre-operative (pre-surgical) imaging with real-time surgical images to provide precision guidance during surgeries, which may reduce errors and improve outcomes.

In various embodiments, real-time surgical imaging systems include one or more energy delivery systems that deliver energy to a region of interest of a patient disposed on an energy transmissive bed top apparatus of an operating table. In some cases, a real-time surgical imaging system includes one or more free-space energy delivery systems (e.g., RF/microwave energy delivery system with an RF/microwave antenna or waveguide). In addition or alternatively, the real-time surgical imaging system may include one or more guided energy delivery systems (e.g., pulse laser energy delivery system with optical fibers coupled to a pulsed laser). In multi-modal imaging examples, a real-time surgical imaging system may include multiple energy delivery systems that can provide different forms of energy (e.g., light/laser, RF/microwave, ultrasound, and other forms of energy) and/or different modes of delivering energy. Some examples of forms of energy and delivery modes that might be employed include delivery of light/laser, RF/microwave, ultrasound, and other forms of energy through free-space or guided mechanisms. The energy may be delivered from above the surgical field and bed top surface of an energy transmissive bed top apparatus or below the bed top surface, which allows for surgical imaging and treatment with minimal interference with surgeries.

### II. Imaging Modalities

In various embodiments, real-time surgical imaging systems can include components of energy delivery system(s) and a detection system that enable one or more imaging modalities.

Some examples of imaging modalities that may be enabled by real-time surgical imaging systems disclosed herein include pulse-echo ultrasound imaging, ultrasound computed tomography (ultrasound CT), photoacoustic tomography (PAT), and/or RF/microwave-induced thermoacoustic tomography (TAT). Each of these techniques offers unique advantages in medical imaging, particularly in providing detailed structural and functional information without the risks associated with ionizing radiation. By combining the strengths of various imaging modalities, certain examples of real-time surgical imaging systems discussed herein can provide detailed, high-contrast images with the safety of non-ionizing radiation, enhancing both diagnostic accuracy and patient safety.

### Pulse-Echo Ultrasound Imaging

Pulse-echo ultrasound imaging is a non-invasive imaging technique that utilizes ultrasound waves to produce images of internal body structures. In this imaging modality, an ultrasonic transducer is used to emit short bursts of sound waves (pulses) into tissues of a region of interest being imaged. These waves reflect off the tissues and return to the ultrasonic transducer(s), creating echoes that can be processed to form images.

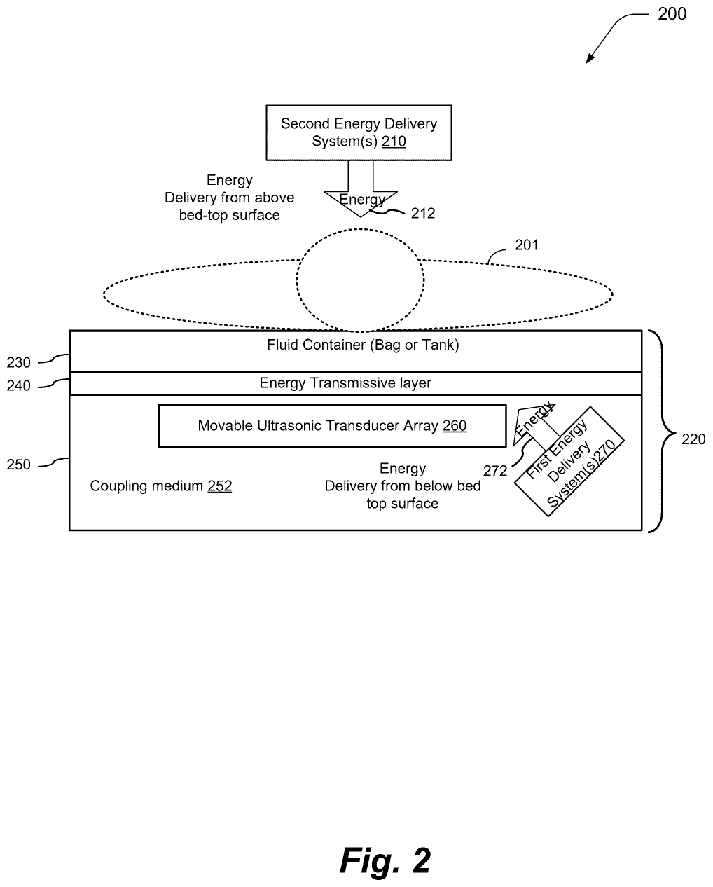

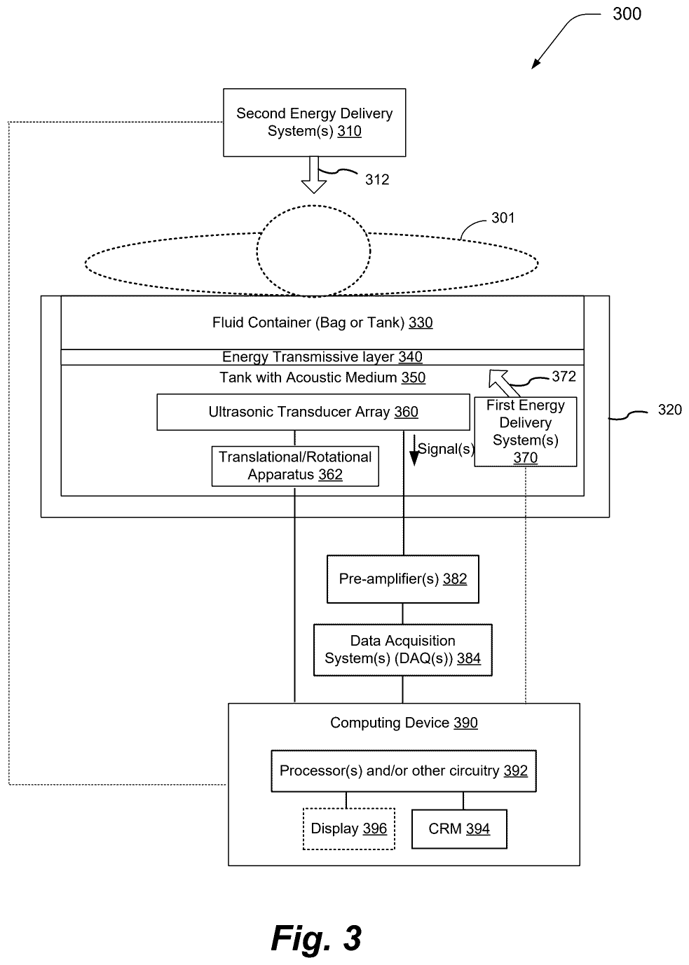

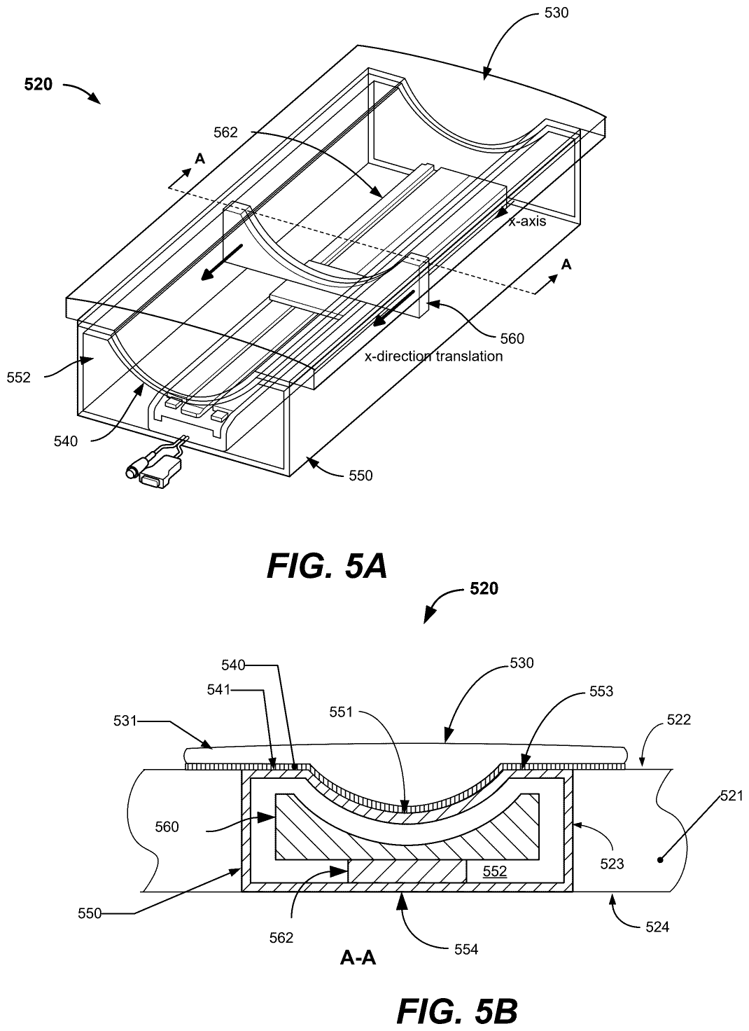

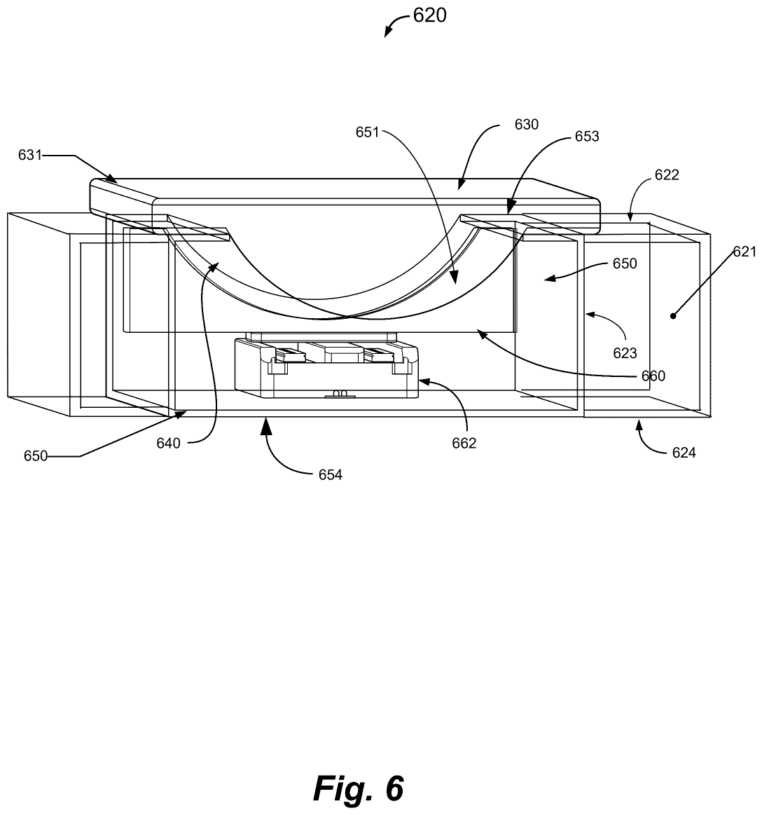

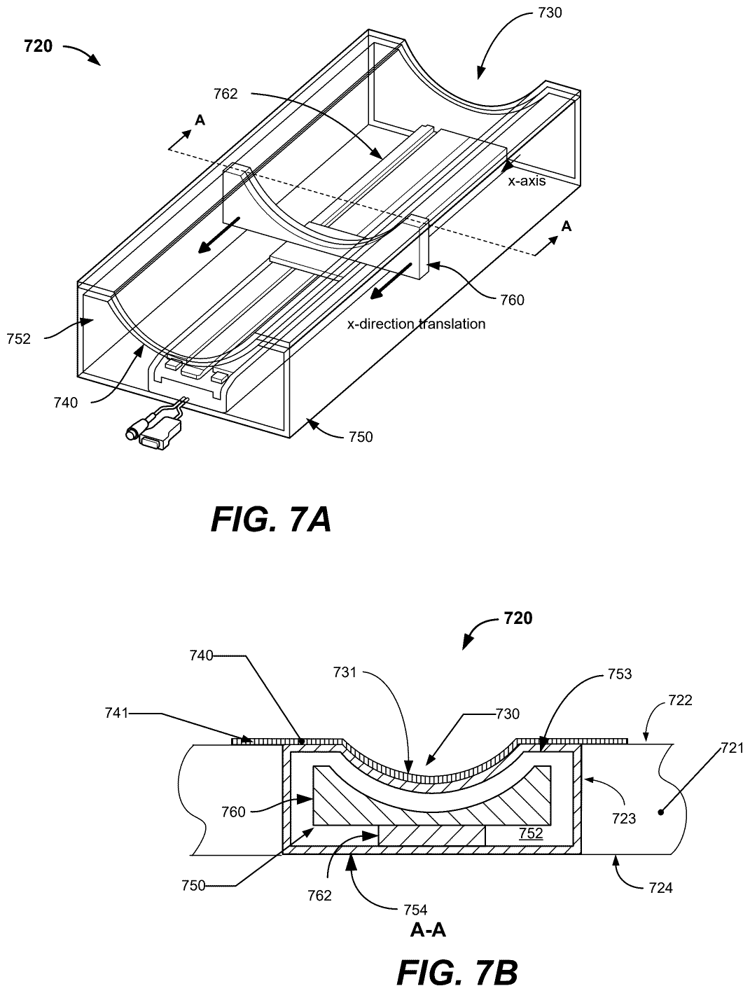

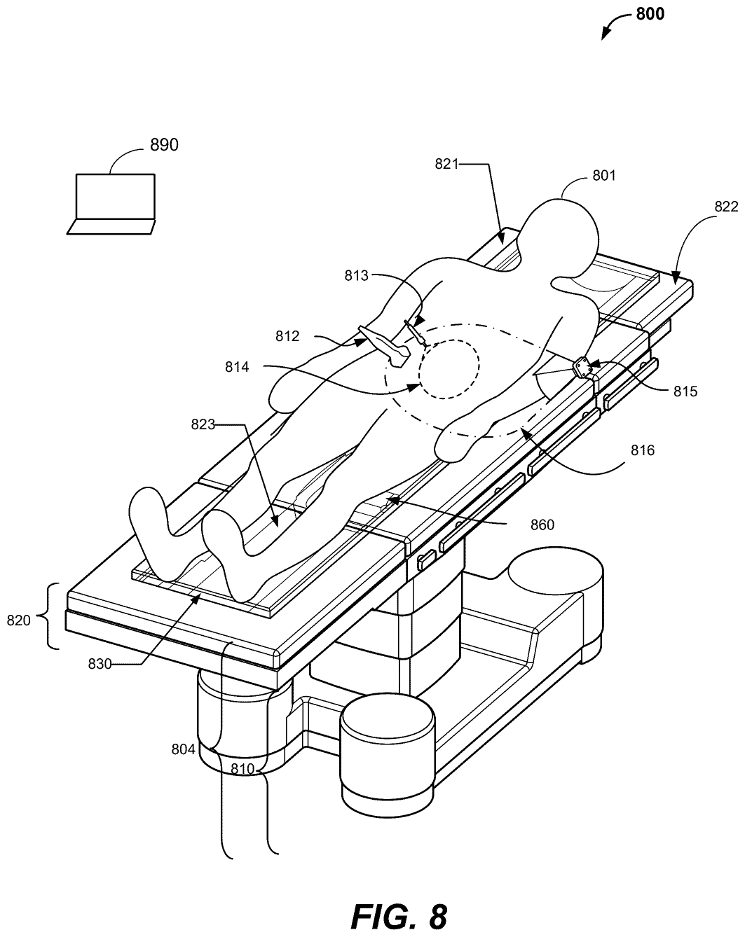

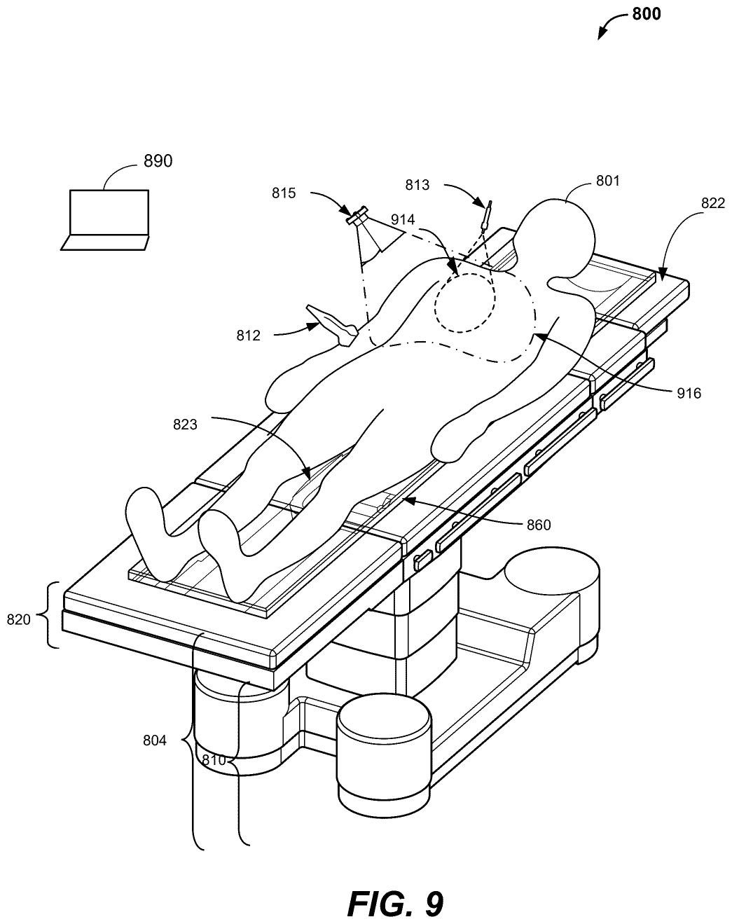

In certain implementations, a real-time surgical imaging system enables pulse-echo ultrasound imaging. In these cases, the real-time surgical imaging system includes one or more sets of ultrasonic transducers (e.g., ultrasonic probe or 1D or 2D ultrasonic transducer array(s)). For example, the real-time surgical imaging system may include an ultrasonic transducer array (e.g., ultrasonic transducer array 260 in FIG. 2, 2D ultrasonic transducer array 560 below bed top surface 522 of energy transmissive bed top apparatus 520 in FIGS. 5A and 5B, or 2D ultrasonic transducer array 760 below bed top surface 722 of energy transmissive bed top apparatus 720 in FIGS. 7A and 7B). A translational/rotational device such as a 1D or 2D translational stage (e.g., translational/rotational apparatus 362 in FIG. 3, X-translational stage 562 in FIGS. 5A and 5B, or X-translational stage 762 in FIGS. 7A and 6B) can be used to translate the ultrasonic transducer array during imaging to acquire multiple images at different cross-sections. In addition or alternatively, the real-time surgical imaging system may include an ultrasonic probe (e.g., ultrasonic probe 812 in FIGS. 8 and 9). In one instance, the ultrasonic transducer array can emit short bursts of energy that are transmitted to the tissues and the waves reflected off the tissues are transmitted back to the ultrasonic transducer array. In another instance example, the real-time surgical imaging system may include an ultrasonic probe or similar device that is used to contact the tissues of the patient, for example, from above the bed-top surface (e.g., ultrasonic probe 812 above bed top surface 822 in FIG. 8) to emit pulses into the tissues and the waves reflected off the tissues are transmitted back to the ultrasonic probe. The probe and/or the transducer array may be translated during imaging. In some instances, a high-resolution ultrasonic probe can be used, such as the X187L5s transducer sold by GE Healthcare.

Some potential advantages of implementations of real-time surgical imaging systems that enable pulse-echo ultrasound imaging include real-time imaging, safety, and portability. Pulse-echo ultrasound provides real-time images which may make it ideal for dynamic studies such as fetal monitoring, cardiac imaging, and guiding interventions. Also, since ultrasound is a form of non-ionizing radiation, it is safe for repeated use, even in sensitive populations such as pregnant women. Moreover, ultrasonic transducer devices such as probes are relatively compact and portable, making them accessible in a wide range of clinical settings.

### Two-Way Ultrasound Imaging

In certain implementations, a real-time surgical imaging system may include an ultrasonic probe and an ultrasonic transducer array (e.g., ultrasonic probe 812 and ultrasonic transducer array 860 in real-time surgical imaging system 800 in FIGS. 8 and 9) that are located outside opposite sides of a surgical bed top apparatus upon which the patient is disposed during imaging. For example, the ultrasonic probe may be placed in contact with the patient from above and the ultrasonic transducer array may be located beneath the bed top surface. In these implementations, the real-time surgical imaging system can perform two-way imaging in both or either of reflection and transmission modes. In one example of reflection mode imaging, the ultrasonic probe emits ultrasound waves (e.g., pulses) and detects ultrasound waves reflected from the patient tissues. The ultrasonic signals detected by the ultrasonic probe can be used to generate pulse-echo reflection image(s). In another example of reflection mode imaging, the ultrasonic transducer array emits ultrasound waves and detects ultrasound waves reflected from the patient tissues. The ultrasonic signals detected by the ultrasonic transducer array can be used to generate pulse-echo reflection image(s). In one example of transmission mode imaging, the ultrasonic probe emits ultrasound waves (e.g., pulses) and the ultrasonic transducer array detects ultrasound waves transmitted through the patient tissues and through at least a portion of the energy transmissive bed top apparatus. The ultrasonic signals detected by the ultrasonic transducer array can be used to generate transmission image(s). In another example of transmission mode imaging, the ultrasonic transducer array emits ultrasound waves and the ultrasonic probe detects ultrasound waves transmitted through patient tissues and through at least a portion of the energy transmissive bed top apparatus. The ultrasonic signals detected by the ultrasonic probe can be used to generate transmission image(s).

### Ultrasound Computed Tomography (Ultrasound CT)

Ultrasound CT is a non-invasive imaging technique that reconstructs cross-sectional images of the tissues using ultrasound data. Unlike pulse-echo ultrasound, which collects echoes from one view, ultrasound CT collects data from multiple angles around the tissues and uses reconstruction algorithms to generate the cross-sectional images.

In certain implementations, a real-time surgical imaging system enables Ultrasound CT imaging. In these cases, the real-time surgical imaging system includes one or more sets of ultrasonic transducers (e.g., transducer(s) in 1D or 2D ultrasonic transducer array(s)). For example, the real-time surgical imaging system may include an ultrasonic transducer array below the bed-top surface of the energy transmissive bed top apparatus (e.g., ultrasonic transducer array 260 in FIG. 2, 2D ultrasonic transducer array 560 below bed top surface 522 of energy transmissive bed top apparatus 520 in FIGS. 5A and 5B, or 2D ultrasonic transducer array 660 below bed top surface 622 of energy transmissive bed top apparatus 720 in FIGS. 7A and 7B). A rotational device (e.g., translational/rotational apparatus 362 in FIG. 3) coupled to the ultrasonic transducer array can be implemented to rotate the ultrasonic transducer array to collect data from multiple angles. The ultrasonic transducer array can deliver energy transmitted through at least a portion of the energy transmissive bed top apparatus to the tissues and the waves reflected off the tissues are transmitted through at least a portion of the energy transmissive bed top apparatus back to the ultrasonic transducer array.

Some potential advantages of implementations of real-time surgical imaging systems that enable ultrasound CT include (i) larger field of view than other forms of ultrasound imaging, (ii) no contact-induced tissue deformation as compared with other forms of ultrasound imaging, (iii) enhanced contrast between different tissue types using both ultrasonic reflection and transmission, and (iv) like other ultrasound-based techniques, ultrasound CT uses non-ionizing radiation, making it safer than X-ray-based CT.

### Photoacoustic Tomography (PAT)

Photoacoustic tomography (PAT) is an imaging technique with the high spatial resolution of ultrasound and high contrast of optical imaging. When implementing PAT imaging, pulsed laser light is absorbed by tissues, causing a rapid thermal expansion that generates ultrasound waves. These ultrasound waves may be detected by ultrasonic transducers to generate acoustic signals that can be used to generate images.

In certain implementations, a real-time surgical imaging system enables PAT. In these cases, the real-time surgical imaging system includes a pulsed laser light delivery system that can deliver laser light from a pulsed laser to a region of interest and one or more sets of ultrasonic transducers (e.g., transducer(s) in 1D or 2D ultrasonic transducer array(s)) for detecting the acoustic waves generated. The pulsed laser light delivery system may deliver the laser light to the region of interest from above or below the bed top surface. For instance, the energy delivery system may include one or more optical fibers (e.g., one or more optical fibers 814 in FIG. 8) in optical communication with a laser light source that can be manipulated to deliver laser light to a region of interest from above the bed top surface and above the surgical field. The one or more sets of ultrasonic transducers may be above or below the bed top surface of the energy transmissive bed top apparatus. For instance, the one or more sets of ultrasonic transducers may be in the form of a ID or 2D ultrasonic transducer array below the bed top surface (e.g., ultrasonic transducer array 260 in FIG. 2, 2D ultrasonic transducer array 560 below bed top surface 522 of energy transmissive bed top apparatus 520 in FIGS. 5A and 5B, or 2D ultrasonic transducer array 760 below bed top surface 722 of energy transmissive bed top apparatus 720 in FIGS. 7A and 7B). A rotational/translational device (e.g., translational/rotational apparatus 362 in FIG. 3, X-translational stage 562 in FIGS. 5A and 5B, or X-translational stage 762 in FIGS. 7A and 7B) such as a rotational/translational stage can be used to translate/rotate the ultrasonic transducer array during imaging to acquire multiple images. In another instance, the one or more sets of ultrasonic transducers may be in the form of an ultrasonic probe that is used to contact the tissues of the patient from above the bed-top surface (e.g., ultrasonic probe 812 above bed top surface 822 in FIGS. 8 and 9). The probe and/or the transducer array may be translated/rotated during imaging.

In some cases, the pulsed laser is configured to generate pulsed light at a near-infrared wavelength or a narrow band of near-infrared wavelengths. For example, the pulsed laser source may be capable of generating near infrared pulses having a wavelength or narrow band of wavelengths (i) in a range from about 700 nm to about 1000 nm, (ii) in a range from about 600 nm to about 1100 nm, (iii) greater than 760 nm, (iv) greater than 1000 nm, or (v) at about 1064-nm. A commercially-available example of a suitable pulsed laser is the PRO-350-10, Quanta-Ray® laser with a 10-Hz pulse repetition rate and 8 ns-12 ns pulse width sold by Spectra-Physics®. The low optical attenuation of 1064 nm light or other near infrared light can be used to deeply penetrate to, e.g., a depth of 4 cm, into biological tissues. Some examples of suitable pulse repetition rates that can be used include about 10-Hz, about 20-Hz, about 50-Hz, and about 100-Hz. In one example, the pulse repetition rate is in a range from about 10-Hz to about 100-Hz. In one aspect, the pulsed laser source may be a tunable narrow-band pulsed laser such as, e.g., one of a quantum cascade laser, an interband cascade laser, an optical parametric oscillator, or other pulsed laser that can be tuned to different narrow bands (e.g., a near-infrared band). The pulsed laser light delivery system may also include one or more optical elements for delivering the laser light from the laser source to the region of interest. Some examples of optical elements that may be included are optical fiber(s), lens(es), optical filter(s), mirror(s), beam steering device(s), beam-splitter(s), relay(s), and/or beam combiner(s)).

One advantage of implementations of real-time surgical imaging systems that enable PAT is high contrast imaging. PAT provides high contrast images based on the optical absorption properties of tissues, making it particularly effective for visualizing blood vessels, hemoglobin concentration, oxygen saturation of hemoglobin, and other chromophores. Another advantage is deep tissue imaging. Unlike traditional optical imaging, which is limited by the scattering of light, PAT can provide detailed images of deeper tissues, overcoming the penetration limits of light. Another potential advantage is the ability to be combined with other imaging modalities for multimodal integration, especially with other ultrasound imaging due to the possibility of sharing the ultrasonic transducer, which can provide complementary information about tissue structure and function.

### RF/Microwave-Induced Thermoacoustic Tomography (TAT)

RF/microwave-induced thermoacoustic tomography (TAT) is an imaging technique that combines rich radio frequency absorption contrast and fine acoustic resolution. When implementing RF/microwave-induced TAT, short pulses of RF or microwave energy are delivered into tissues, leading to absorption-specific thermoelastic expansion released as thermoacoustic waves. These thermoacoustic waves can be detected by ultrasonic transducers and the signals can be used to reconstruct images of the absorption profile. The resulting images have contrast relating to water and ionic content and provide mm-scale resolution of biological structures within the tissues.

In certain implementations, a real-time surgical imaging system enables TAT imaging. In some of these cases, the real-time surgical imaging system has an energy delivery system with a radio frequency (RF)/microwave antenna (e.g., microwave antenna 816 in FIG. 8 and microwave antenna 916 in FIG. 9) that can irradiate a region of interest of the patient and/or attenuate stray radio frequency signals. In other cases, the real-time surgical imaging system has an energy delivery system with a waveguide that can transmit RF/microwave energy from the energy source to the surgical site with minimal loss. Waveguides are particularly useful for delivering energy over longer distances or when the energy needs to be confined to a specific path. The waveguide structure ensures that the energy is efficiently transmitted with minimal dispersion, maintaining the integrity of the signal. The real-time surgical imaging system also includes one or more sets of ultrasonic transducers may be above or below the bed top surface of the energy transmissive bed top apparatus. For instance, the one or more sets of ultrasonic transducers may be in the form of a 1D or 2D ultrasonic transducer array below the bed top surface (e.g., ultrasonic transducer array 260 in FIG. 2, 2D ultrasonic transducer array 560 below bed top surface 522 of energy transmissive bed top apparatus 520 in FIGS. 5A and 5B, or 2D ultrasonic transducer array 760 below bed top surface 722 of energy transmissive bed top apparatus 720 in FIGS. 7A and 7B). A rotational/translational device (e.g., translational/rotational apparatus 362 in FIG. 3, X-translational stage 562 in FIGS. 5A and 5B, or X-translational stage 762 in FIGS. 7A and 7B) such as a rotational/translational stage can be used to translate/rotate the ultrasonic transducer array during imaging to acquire multiple images. In another instance, the one or more sets of ultrasonic transducers may be in the form of an ultrasonic probe that is used to contact the tissues of the patient from above the bed-top surface (e.g., ultrasonic probe 812 above bed top surface 822 in FIGS. 8 and 9). The probe and/or the transducer array may be translated/rotated during imaging.

Implementations that enable TAT offer advantages including high tissue contrast, deep tissue penetration, and non-ionizing radiation. For example, TAT leverages the differences in the dielectric properties of tissues to produce high-contrast images, making it useful for detecting tumors and other abnormalities. Also, RF/microwave energy can penetrate deep into tissues, allowing for the imaging of structures that are beyond the reach of optical methods. Moreover, TAT uses non-ionizing radiation, making it a safer alternative to X-ray and other ionizing imaging techniques.

### III. Examples of Real-time Surgical Imaging Systems

In various embodiments, real-time surgical imaging systems include a surgical table with an energy transmissive surgical bed top apparatus having components with a top surface (sometimes referred to herein as a bed top surface) that are designed to enable transmission of ultrasonic, optical, RF/microwave energy, and/or other forms of energy. In certain implementations, these real-time surgical imaging systems include a 1D/2D ultrasonic transducer array mounted on a translation stage (e.g., x-translational stage, X-Y translational stage, articulating arm, etc.), capable of precise movement and tilting to target specific anatomical regions. Free-space energy delivery mechanisms, such as lasers and RF/microwave emitters, may be integrated above and below the surgical table, allowing non-contact energy delivery above the surgical field or through the bed-top. In certain implementations, real-time surgical imaging systems may also incorporate energy-enabled surgical tools, including scalpels and cautery devices with integrated optical fibers, ultrasonic transducers, and RF/microwave components. These tools may be used to deliver energy directly to the surgical site, functioning as waveguides or antennas depending on the energy modality.

In various embodiments, real-time surgical imaging systems implement elastic co-registration of pre-surgical images (e.g., from CT, MRI, US, PET, etc.) with real-time intraoperative images. This co-registration is achieved using advanced image processing algorithms that align the pre-surgical data with the patient's current anatomical state. The real-time images may be displayed on high-resolution monitors, providing continuous guidance during the surgery.

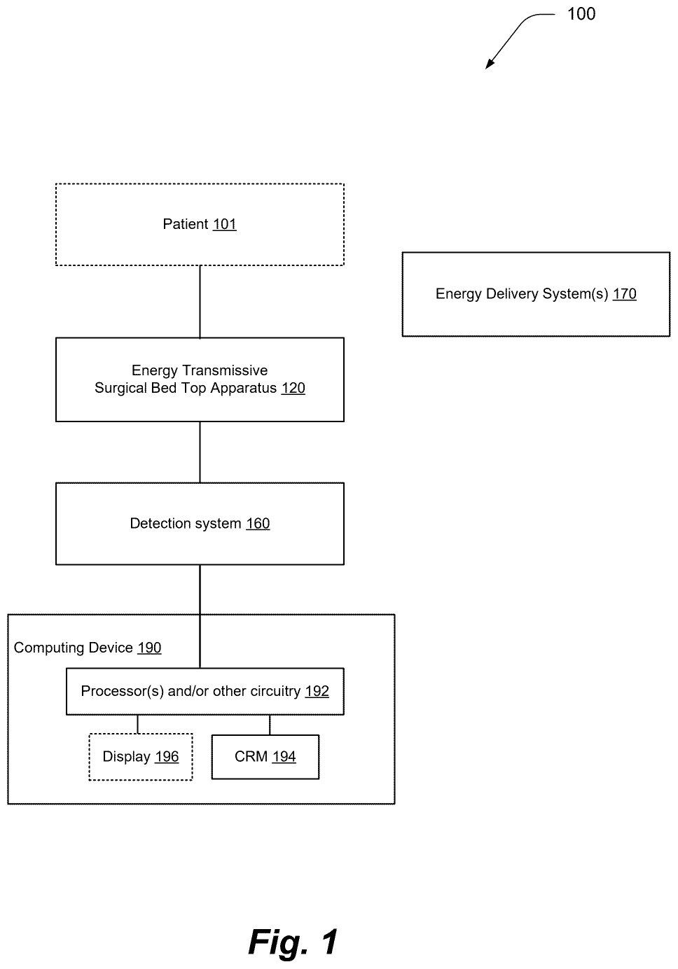

FIG. 1 is a block diagram of components of a real-time surgical imaging system 100 with an energy transmissive surgical bed top apparatus 120, according to certain implementations. The real-time surgical imaging system 100 includes one or more energy delivery systems 170 having one or more energy delivery components (e.g., optical fiber(s), radio frequency antenna or waveguide, ultrasonic transducer(s), etc.) that can deliver energy to a region of interest of a patient 101 disposed on top of the energy transmissive surgical bed top apparatus 120. The energy delivery component(s) are in communication with corresponding energy source(s) which may be components of the real-time surgical imaging system 100 or may be separate from the real-time surgical imaging system 100. For example, the real-time surgical imaging system 100 may include one or more optical fibers in optical communication with a separate remotely located pulse laser. According to various implementations, the one or more energy delivery systems 170 may implement free space delivery of energy and/or guided energy delivery. The one or more energy delivery systems 170 may have one or more components for delivering energy from above a bed top surface of the energy transmissive surgical bed top apparatus 120 (e.g., bed top surface 522 in FIG. 5 or bed top surface 722 in FIG. 7), below the bed top surface, or to the energy transmissive surgical bed top apparatus 120.

The real-time surgical imaging system 100 also includes a detection system 160 in communication with the energy transmissive surgical bed top apparatus 120 to detect energy waves from the region of interest that may pass through the energy transmissive surgical bed top apparatus 120. The detection system 160 includes one or more energy detecting devices (e.g., ultrasonic transducer array, ultrasonic probe, etc.) for detecting energy from above, below, and/or to the side of the bed top surface. In some implementations, the detection system 160 may include a rotational/translational apparatus coupled to a detector to translate/rotate the detector during imaging. In various examples, the rotational/translational apparatus may have one (1) to six (6) degrees of freedom (DOF) of translation and rotation. For example, in one implementation, a rotational/translational apparatus may be a translational stage for translating along a single axis (e.g., X-translational stage 562 in FIGS. 5A and 5B or X-translational stage 762 in FIGS. 7A and 7B). In another implementation, a rotational/translational apparatus may be a translational stage for translating along two axes (e.g., X-Y translational stage). In another implementation, the rotational/translational apparatus may include an articulating arm, on which the detector is mounted, that can rotate and translate the detector along six DOF.

The energy transmissive surgical bed top apparatus 120 includes at least a portion through which energy from the energy delivery system(s) 170 can pass to the patient 101 and/or from the patient 101 to the detection system 160. The illustration is shown at an instant in time during an imaging operation. It would be understood that at other times, the patient 101 may not be disposed on the energy transmissive surgical bed top apparatus 120 and is considered optional as denoted by the dotted line.

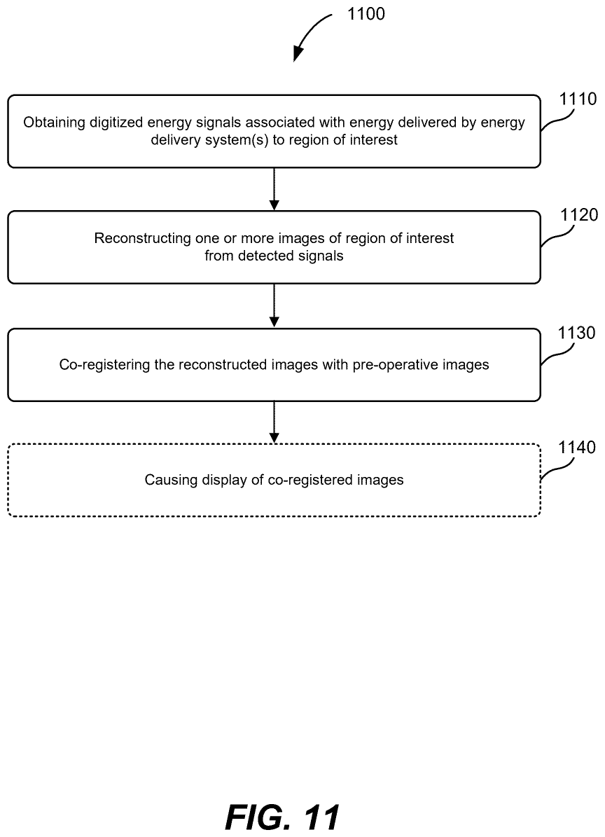

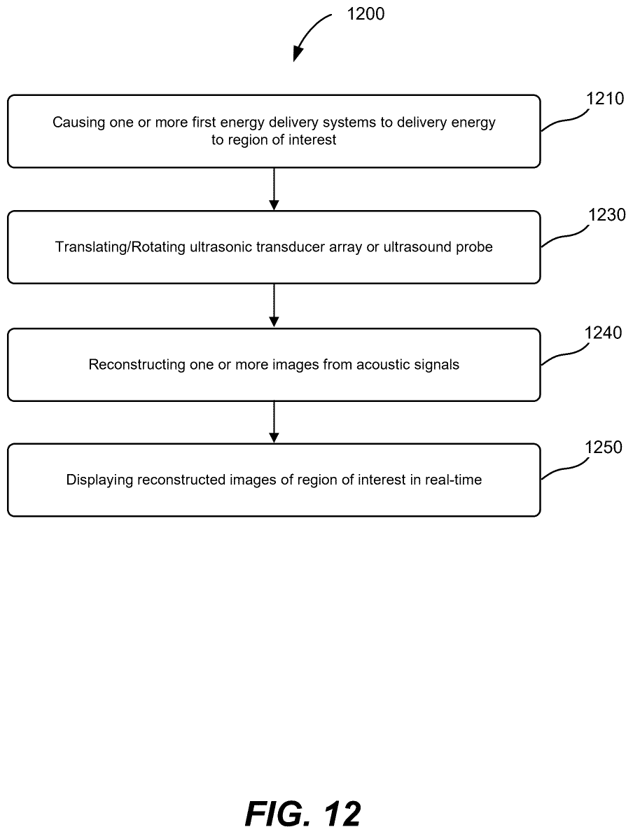

The real-time surgical imaging system 100 also includes a computing device 190 with includes one or more processors and/or other circuitry 192 in electrical communication with detection system 160 to receive digitized energy signals (e.g., acoustic data). The one or more processors and/or other circuitry 192 execute instructions to perform image processing operations and other operations of methods disclosed herein such as the method depicted in FIGS. 11 and 12. The computing device 190 also includes a computer readable media 194 and an optional (denoted by dotted line) display 196 in electrical communication with the one or more processors and/or other circuitry 192.

In various implementations, a real-time surgical imaging system includes an energy transmissive surgical bed top apparatus with at least a portion through which energy can pass through from above the surgical bed top surface to components below the surgical bed top surface and energy can pass through from components below the surgical bed top surface to a patient disposed above the surgical bed top surface. The portion through which energy can pass may include various energy transmissive components such as a fluid bag or tank, one or more energy transmissive layer, and a tank with a coupling medium such as water or mineral oil. It would be understood that certain other portions of the energy transmissive surgical bed top apparatus may not allow energy to pass through. For example, the energy transmissive surgical bed top apparatus may have a portion with an aperture (e.g., aperture 723 in FIGS. 7 and 8) and one or more one or more components with low impedance to transmit energy with minimal absorption. An (outer) portion (e.g., outer portion 521 in FIG. 5, outer portion 621 in FIG. 6, outer portion 721 in FIG. 7, or outer portion 821 in FIG. 8) around the aperture may have higher impedance and absorb energy in some cases.

In various embodiments, a real-time surgical imaging system includes one or more energy transmissive layers. An energy transmissive layer generally refers to a material layer with properties providing minimal attenuation to the energy being passed through the energy transmissive surgical bed top apparatus. For example, the energy transmissive layer(s) may have low acoustic impedance properties to allow for transmission of acoustic waves with minimal attenuation. Some examples of materials with low acoustic impedance include water, acoustic gel, mineral oil, etc. As another example, the energy transmissive layer(s) may have properties that enable transmission of light and RF/microwave energy. In one aspect, the energy transmissive layer has material properties that closely match the acoustic impedance of water or acoustic gel to minimize reflection and artifacts. In one implementation, the energy transmissive layer is a layer of polymethylpentene (PMP). A commercially-available example of PMP is TPX® PMP sold by Mitsui Chemicals. In another implementation, the energy transmissive layer is a plastic. Various thicknesses of the energy transmissive layer may be used. In certain implementations, an energy transmissive layer is located between a fluid bag or tank and another tank with water or mineral oil. In other cases, the energy transmissive layer may be a layer of the fluid bag or tank or of the additional tank with water or mineral oil. In some cases, the energy transmissive layer may provide support to at least a portion of the weight of the body of the patient. For example, the energy transmissive layer 540 in FIG. 5 has edge portions 541 that overhang on top of the bed top surface 522 outside the aperture 523. The edge transmissive layer 540 also has a curved portion on top of the curved surface 551 of the tank 550. The weight of a patient lying on top of the fluid bag 530 may be supported at least partially by the energy transmissive layer 540 and the edges of the bed top surface 522 outside the aperture 523.

In various embodiments, a real-time surgical imaging system includes a fluid container such as a fluid bag (e.g., a water bag or a bag containing mineral oil or the like) upon which the patient is disposed during imaging or a fluid tank (e.g., water tank or tank containing mineral oil or the like) within which a portion of the body of the patient is disposed during imaging. In some embodiments, the real-time surgical imaging system includes a fluid bag placed at least partially above the bed top surface. For example, the fluid bag 530 in FIGS. 5A and 5B has an edge portion that is on top of the bed top surface 522 and a central portion that lies below the bed top surface 522. During image acquisition, the patient is located on top of the fluid bag and a thin layer of water or ultrasonic coupling gel is applied between the fluid bag and the patient proximal the region of interest being imaged. In some cases, the wall of the fluid bag is made of a material that is soft, flexible, and transmissive to both ultrasound and RF/microwave energy, ensuring minimal attenuation and efficient transmission of energy. Some examples of materials that can be used include a thin polyethylene (PE) film, a polyurethane (PU) film (e.g. medical-grade TPU), a polyvinyl chloride (PVC) film which is soft, plasticized, a silicone elastomer sheet, a mylar (e.g., biaxially oriented PET) film if flexibility is acceptable, a low-density polyethylene (LDPE) or ethylene-vinyl acetate (EVA) film for higher softness. A sheet with an open segment that allows access to at least an area of the patient's skin proximal the region of interest for imaging may be placed over the fluid bag during image acquisition. A disposable plastic film may be placed over the open segment to maintain sterility and optimize the transmission of both ultrasound and other energy. Water or acoustic gel may be used in any space between the plastic film and the patient's skin to ensure efficient transmission of acoustic waves. In other embodiments, the real-time surgical imaging system may include a fluid tank (e.g., tank containing mineral oil or the like). For example, the fluid tank 730 in FIGS. 7A and 7B is formed by the outer surface at the central curved portion of the energy transmissive layer 740. In another embodiment, the fluid tank may be a separate component coupled to the energy transmissive layer. The fluid tank can be used to acoustically couple acoustic waves between the patient and other components of the energy transmissive surgical bed top apparatus. The water temperature within the fluid bag or tank may be adjusted for patient comfort, for example, with one or more integral heating elements. This is particularly beneficial for longer surgical procedures, where maintaining a comfortable temperature can help prevent hypothermia or discomfort.

In various implementations, real-time surgical imaging systems include one or more energy delivery systems that can deliver energy to a region of interest of a patient located on top of an energy transmissive surgical bed top apparatus. The energy delivery system(s) may have one or more components for delivering energy from above or to the side of a bed top surface of the energy transmissive surgical bed top apparatus (e.g., bed top surface 522 in FIG. 5 or bed top surface 722 in FIG. 7), from below the bed top surface, or to the energy transmissive surgical bed top apparatus. For example, a real-time surgical imaging system may include one or more energy delivery systems (e.g., microwave antenna 716 or optical fiber(s) 714 in FIG. 7 or microwave antenna 816 or optical fiber(s) 814 in FIG. 8) that can deliver energy from above or to the side of the bed top surface of the surgical table with no or minimal interference with surgeries for either imaging or therapy. This example implementation may be particularly useful in scenarios where direct contact with the tissue is impossible or undesired. In some cases, for targeted intervention and simultaneous imaging without the need for physical contact with the tissue, the energy can be precisely focused. Implementations that deliver energy from below the bed top surface below the patient or from the side may advantageously reduce the risk of interference with the surgical field, maintaining a clear workspace for the surgical team while still providing essential imaging and treatment capabilities. Also, these implementations may allow for efficient and targeted energy application directly to the patient's body, enhancing both imaging and therapeutic outcomes.

In certain implementations, an energy delivery system may include one or more articulating arms and/or optical elements that can enable free-space delivery of light energy from above, to the side, or from below the bed top surface of the energy transmissive surgical bed top apparatus to the region of interest being imaged. For example, an energy delivery system may include at least one articulating arm with one or more optical elements such as mirror(s) or lens(es) are in optical communication with a laser source and can be used to direct light or laser beams from the laser source onto the region of interest. Implementations that include articulating arm(s) may offer a high degree of maneuverability, enabling the surgeon to adjust the angle and focus of the light beam as needed during the imaging procedure. The use of articulated arms allows for non-contact high-energy delivery, which may reduce the risk of contamination and maintain a clear surgical field. In some cases, an energy delivery system may include one or more optical fibers (e.g., fiber bundle with multiple optical fibers) or free-space delivery systems integrated into the energy transmissive surgical bed top apparatus. The optical fiber(s) or free-space delivery system(s) are in optical communication with a laser source or other light-based energy source to enable directly light into the tissue for therapeutic or imaging. The optical fiber(s) can be maneuvered to deliver light directly to the region of interest (e.g., surgical site). The one or more optical fibers can be bundled together to form flexible conduits that transmit light with minimal loss, allowing for precise illumination or laser energy delivery. The flexibility of fiber bundles can make them ideal for accessing hard-to-reach areas within the surgical field. Implementations with optical energy delivery systems such as delivery systems having articulating arms with optical components for delivering light and delivery systems having optical fiber(s) can be used to target laser energy that enables photoacoustic imaging and laser-assisted surgeries. In some cases, certain materials of the energy transmissive surgical bed top apparatus may be optically transparent, ensuring light from a light energy delivery system can be transmitted through the materials to/from the patient efficiently without significant loss.

In certain implementations, an energy delivery system may include one or more RF/microwave antennas or one or more waveguides that can deliver RF/microwave energy from an RF/microwave energy source to the region of interest. In some cases, the antenna(s) or waveguide(s) may deliver energy from above or to the side of a bed top surface of the energy transmissive surgical bed top apparatus to the region of interest being imaged. In other cases, the antenna(s) or waveguide(s) may deliver energy from below the bed top surface of the energy transmissive surgical bed top apparatus to the region of interest. Some examples of suitable RF sources include a pulsed cavity magnetron, a spark gap generator, a solid-state amplifier. a traveling wave tube amplifier and a klystron. In examples that implement one or more RF/microwave antennas, the RF/microwave antennas can be designed to deliver RF/microwave energy for specific frequency ranges (e.g., a range of about 0.3-3.0 GHz or a frequency of about 1.1 GHZ) to the surgical site. RF/microwave antennas designed for delivery energy in specific frequency ranges can be used to focus the RF/microwave energy on a targeted area enabling localized heating, ablation, and/or imaging. In certain instances, an RF/microwave antenna may be integrated into a surgical tool, such as a scalpel or a cautery device, to enable delivering energy precisely where it is needed. In examples that implement one or more waveguides, the energy delivery system may transmit RF/microwave energy from the energy source to the surgical site with minimal loss. Waveguides are particularly useful for delivering energy over longer distances or when the energy needs to be confined to a specific path. The waveguide structure may ensure that the energy is efficiently transmitted with minimal dispersion, maintaining the integrity of the signal. Delivering RF/microwave energy enables procedures such as microwave ablation, where precise control over the energy delivery is necessary to achieve the desired therapeutic effect. In some cases, materials and components of the energy transmissive surgical bed top apparatus are designed to be RF/microwave transmissive, allowing RF/microwave energy to pass through the materials and into the patient's body with minimal attenuation. The ability to deliver RF/microwave energy from below the bed top surface provides additional flexibility in treatment options, including heating, ablation, or RF/microwave-induced thermoacoustic imaging, depending on the surgical requirements.

In certain implementations, an energy delivery system and/or detection system may include one or more ultrasonic probes that can deliver and/or receive ultrasonic waves from above or to the side of a bed top surface of the energy transmissive surgical bed top apparatus to the region of interest being imaged. In some cases, the one or more ultrasonic probes may be used to transmit ultrasonic waves and the detection system includes an ultrasonic transducer array below or to the side of the energy transmissive surgical bed top apparatus that can receive transmitted ultrasonic waves. The received ultrasonic waves can be used as input to an ultrasound tomography procedure to generate 3D images of the region of interest. Implementations that use an ultrasonic probe to deliver energy may advantageously enable higher resolution images than an example that uses an ultrasonic transducer array to deliver energy. In these implementations, an acoustic coupling medium (e.g., water or acoustic gel) may be provided between the tissue and the ultrasonic probe during the data acquisition procedure. The one or more ultrasonic probes may be handheld or may be mounted on an articulating arm of, for example, a robotic system. In some of these implementations, the one or more ultrasonic probes may be high-resolution or microscopic ultrasonic probes that are equipped with high-frequency ultrasonic transducers for detailed, localize imaging such as with photoacoustic imaging.

In certain implementations, an energy delivery system and/or detection system may include one or more ultrasonic transducer elements that can deliver and/or receive ultrasonic waves from below or to the side of a bed top surface of the energy transmissive surgical bed top apparatus to the region of interest being imaged. The one or more ultrasonic transducer elements may be in the form of a ID array or a 2D array, for example. In these implementations, the energy transmissive surgical bed top apparatus may include one or more components with acoustic coupling medium between the patient and the one or more ultrasonic transducer elements during a data acquisition procedure. The one or more ultrasonic transducer elements may be immersed in a coupling medium during data acquisition. For example, the one or more ultrasonic transducer elements may be immersed in an acoustic medium (e.g., water or mineral oil) in a tank below the bed top surface (e.g., tank 250 in FIG. 2, tank 550 in FIG. 5, or first tank 750 in FIG. 7). One or more layers of acoustic transmissive material may be placed between the tank and the patient. For example, a bag or tank of water (e.g., fluid bag 530 in FIG. 5 or fluid tank 730 in FIG. 7) may be placed between the patient and the tank. This placement of layers of acoustic mediums helps ensure transmission of acoustic waves into and/or from the body of the patient, facilitating high-resolution imaging or focused ultrasound therapy. By mounting the one or ultrasonic transducer elements to a translational/rotational apparatus, the positioning of the ultrasonic transducer elements may be adjusted enabling focusing on specific regions of interest within the body.

In various implementations, a real-time surgical imaging system may include one or more components with a coupling medium such as water or mineral oil, which enables transmission of ultrasound waves as well as RF/microwaves and/or light. Water and mineral oil may be used advantageously for their acoustic and other properties that may minimize loss of the relevant energy being transmitted. In some cases, an ultrasonic transducer array may be mounted on a translational/rotational apparatus (e.g., translational/rotational apparatus 362 in FIG. 3, translational stage, etc.) to enable translation/rotation to be moved and/or tilted towards specific regions of interest. In some cases, the translational/rotational apparatus may move the ultrasonic transducer array to various positions and maintain the ultrasonic transducer array at each position for a period of time (e.g., about 10 seconds, about 15 seconds, about 20 seconds, in a range of 10-20 seconds). An example of a mechanism for linear movement in one direction is a KR4610D linear stage made by THK America, Inc.

In some implementations, a real-time surgical imaging system may include one or more energy-enabled surgical tools. For example, a real-time surgical imaging system may include one or more scalpels and/or cautery devices that are integrated with one or more optical fibers, one or more ultrasonic transducers, and/or one or more RF microwave antennas or waveguides. Alternatively, these energy-enabled surgical tools can function as waveguides for ultrasound or antennas for RF/microwave energy, delivering energy precisely at the cutting edge. These energy-enabled surgical tools can deliver energy for either imaging or therapy or both.

In some cases, materials of the energy transmissive surgical bed top apparatus may be optically transparent, ensuring light from a light energy delivery system can be transmitted to/from the patient efficiently without significant loss. In some cases, materials and components of the energy transmissive surgical bed top apparatus are designed to be RF/microwave transmissive, allowing RF/microwave energy to pass through the materials and into the patient's body with minimal attenuation. The ability to deliver RF/microwave energy from below the bed top surface provides additional flexibility in treatment options, including heating, ablation, or RF/microwave-induced thermoacoustic imaging, depending on the surgical requirements.

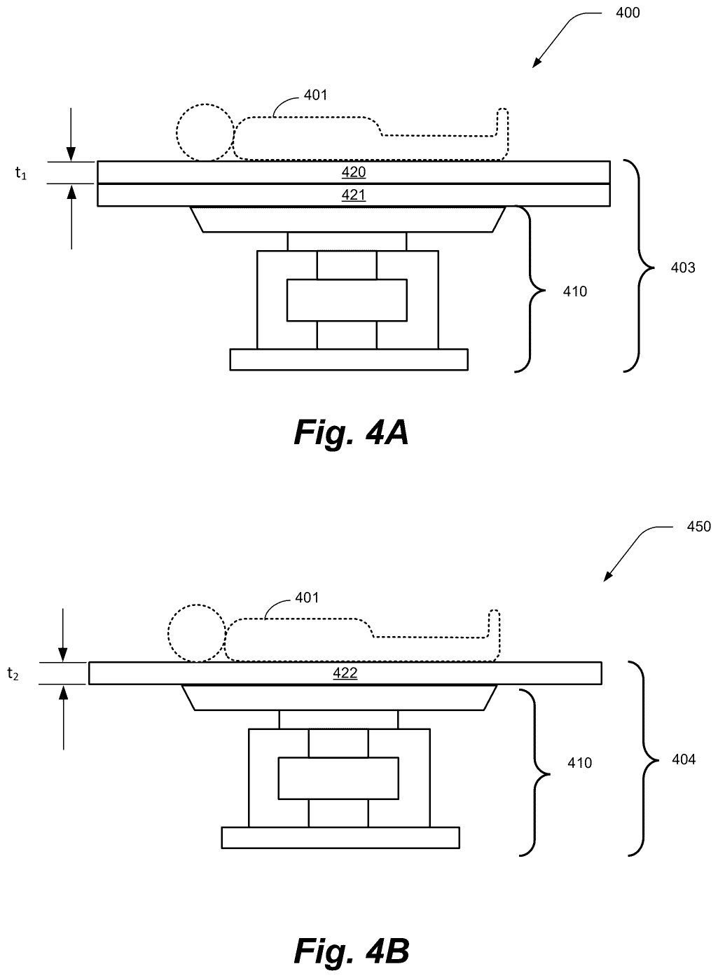

FIG. 2 is a schematic drawing of an example of components of a real-time surgical imaging system 200 including an energy transmissive surgical bed top apparatus 220, according to various implementations. Energy transmissive surgical bed top apparatus 220 may be configured to be coupled to a support assembly (e.g., support assembly 410 in FIGS. 4A and 4B or support assembly 710 in FIGS. 7 and 8) of a surgical table or coupled to an existing surgical bed top (e.g., existing surgical bed top 421) that is coupled to the support assembly. In some cases, the support assembly may be operable to translate/rotate with multiple degrees of freedom (DOF). The illustration is shown at an instant in time during an imaging operation when a patient 201 is disposed on top of the energy transmissive surgical bed top apparatus 220. It would be understood that at other times, the patient 201 may not be disposed on the energy transmissive surgical bed top apparatus 220 and the patient 201 is considered optional included as denoted by the dotted line.

FIG. 4A is a drawing of a side view of components of a real-time surgical imaging system 400 with an example of an energy transmissive surgical bed top apparatus 420 configured to couple to and/or decouple from an existing surgical bed top 421, according to implementations. In this example, the energy transmissive surgical bed top apparatus 420 may be in a modular format configured for installation by placing the energy transmissive surgical bed top apparatus 420 on top of the upper surface of the existing surgical bed top 421 to form the surgical table 403. A locking mechanism or other coupling mechanism may be used to secure the energy transmissive surgical bed top apparatus 420 in place. The energy transmissive surgical bed top apparatus 420 may also be removable. In one aspect, the thickness, t₁, of the energy transmissive surgical bed top apparatus 420 may be less than 20 inches. In one aspect, the thickness, t₁, of the energy transmissive surgical bed top apparatus 420 may be about 10 inches.

FIG. 4B is a drawing of a side view of components of a real-time surgical imaging system 400 with an example of an energy transmissive surgical bed top apparatus 422 configured to couple to a standard support assembly 410, according to implementations. In this example, the energy transmissive surgical bed top apparatus 422 replaces the existing surgical bed top (e.g., existing surgical bed top 421 in FIG. 4A) to form the surgical table 404. In one aspect, the thickness, t₂, of the energy transmissive surgical bed top apparatus 422 may be less than 20 inches. In one aspect, the thickness, t₂, of the energy transmissive surgical bed top apparatus 420 may be about 10 inches.

Returning to FIG. 2, the real-time surgical imaging system 200 includes one or more first energy delivery systems 270 with energy delivery components (e.g., ultrasonic transducer array, RF/microwave antenna, ultrasonic probe, optical fiber(s), etc.) that can deliver energy 272 from below a bed top surface. The real-time surgical imaging system 200 also includes one or more second energy delivery systems 270 with energy delivery components (e.g., ultrasonic transducer array, RF/microwave antenna, ultrasonic probe, optical fiber(s), etc.) that can deliver energy 212 from above a bed top surface or from the side of the energy transmissive surgical bed top apparatus 220. In some cases, the first energy delivery system(s) 270 and/or the second delivery system(s) 210 may deliver energy that does not require contact (free space delivery) with the patient 201 such as light/laser, RF/microwave, and other forms of energy that do not require contact. In addition or alternatively, the first energy delivery system(s) and/or the second delivery system(s) may deliver energy that requires contact such as ultrasound waves and other forms of energy that require contact.

The energy transmissive surgical bed top apparatus 220 includes a fluid container 230 such as a fluid bag or fluid tank and an energy transmissive layer 240. In some implementations, the fluid container 230 and/or the energy transmissive layer 240 are located above the surgical bed top surface. In other implementations, at least a portion of the fluid container 230 is located below the surgical bed top surface. For example, the upper surface of the fluid container 230 may be at or below the surgical bed top surface. The energy transmissive surgical bed top apparatus 220 also includes a tank 250 with a coupling medium 252 such as water, mineral oil, or other liquid with properties for low absorption of light, sound, and RF/microwave energy. In some implementations, at least a portion of the tank 250 is located below the surgical bed top surface.

The real-time surgical imaging system 200 also includes a detection system including a movable ultrasonic transducer array 260 for detecting ultrasonic waves and/or transmitting ultrasonic energy. In this example, the movable ultrasonic transducer array 260 is located within an inner volume of the tank 250 of the energy transmissive surgical bed top apparatus 220. In an alternative implementation, the detection system may include alternate or additional detectors such as an ultrasonic probe located above or to the side of the surgical bed top surface. In some instances, the movable ultrasonic transducer array 260 may be a 1D or 2D ultrasonic transducer array mounted to a rotational/translational apparatus (e.g., X-translational stage 562 in FIGS. 5A and 5B, X-translational stage 762 in FIGS. 7A and 7B, and X-Y stage, an articulating arm configured to rotate/translation in 1-6 DOF, etc.) that can translate/rotate the ultrasonic transducer array during image acquisition. According to one aspect, the rotational/translational apparatus may be able to rotate and translate with one (1) to six (6) degrees of freedom (DOF).

It would be understood that the energy transmissive surgical bed top apparatus 220 may include one or more additional materials or features without departing from the scope of the disclosure. For example, there may be an acoustic coupling material and a plastic protective film located between the fluid container 230 and the patient 201 during imaging. In one aspect, the fluid container 230, energy transmissive layer 240 and tank 250 have material properties of low acoustic impedance and features that enable transmission of acoustic energy with minimal absorption and low attenuation.

The real-time surgical imaging system 200 may also include a computing device with one or more processors and/or other circuitry in electrical communication with the movable ultrasonic transducer array 260 and any other detectors of the detection system to receive digitized energy signals (e.g., acoustic data). The one or more processors and/or other circuitry may execute instructions to perform image processing operations and other operations of methods disclosed herein. The computing device also includes a computer readable media and an optional display in electrical communication with the one or more processors and/or other circuitry.

FIG. 3 is a schematic drawing of an example of components of a real-time surgical imaging system 300 including an energy transmissive surgical bed top apparatus 320, according to various implementations. Energy transmissive surgical bed top apparatus 320 may be configured to be coupled to a support assembly of a surgical table or coupled to an existing surgical bed top that is coupled to the support assembly. In some cases, the support assembly may be operable to translate/rotate with multiple degrees of freedom (DOF). The illustration is shown at an instant in time during an imaging operation when a patient 301 is disposed on top of the energy transmissive surgical bed top apparatus 320. At other times, the patient 301 may not be disposed on the energy transmissive surgical bed top apparatus 320 and the patient 301 is considered optional included as denoted by the dotted line.

The energy transmissive surgical bed top apparatus 320 includes a fluid container 330 such as a fluid bag or fluid tank, an energy transmissive layer 340, a tank 350, a detection system including an ultrasonic transducer array 360, and a translational/rotational apparatus 370. The energy transmissive surgical bed top apparatus 320 also includes one or more one or more first energy delivery systems 370 with energy delivery components (e.g., ultrasonic transducer array, RF/microwave antenna, ultrasonic probe, optical fiber(s), etc.) that can deliver energy 372 from below a bed top surface. In this example, the first energy delivery system(s) 370 are within the tank 350. In other implementations, one or more of the first energy delivery systems 370 may be outside the tank 350.

In some implementations, the fluid container 330 and/or the energy transmissive layer 340 are located above the surgical bed top surface. In other implementations, at least a portion of the fluid container 330 is located below the surgical bed top surface. The tank 350 includes a coupling medium 352 such as water, mineral oil, or other liquid with properties for low absorption of light, sound, and RF/microwave energy. In some implementations, at least a portion of the tank 350 is located below the surgical bed top surface.

The real-time surgical imaging system 300 also includes one or more second energy delivery systems 370 with energy delivery components (e.g., ultrasonic transducer array, RF/microwave antenna, ultrasonic probe, optical fiber(s), etc.) that can deliver energy 312 from above a bed top surface or from the side of the energy transmissive surgical bed top apparatus 320. In some cases, the first energy delivery system(s) 370 and/or the second delivery system(s) 310 may deliver energy that does not require contact (free space delivery) with the patient 301 such as light/laser, RF/microwave, and other forms of energy that do not require contact. In addition or alternatively, the first energy delivery system(s) and/or the second delivery system(s) may deliver energy that requires contact such as ultrasound waves and other forms of energy that require contact.

The real-time surgical imaging system 300 includes a detection system that includes the ultrasonic transducer array 360 (e.g., 1D or 2D ultrasonic transducer array) for detecting ultrasonic waves and/or delivering ultrasonic energy and the translational/rotational apparatus 362 (e.g., X-translational stage, X-Y translational stage, articulating arm for rotating/translating in 1-6 DOF, etc.). In this example, the movable ultrasonic transducer array 360 and translational/rotational apparatus 362 are located within an inner volume of the tank 250 of the energy transmissive surgical bed top apparatus 320. In an alternative implementation, the detection system may include alternative or additional detectors such as an ultrasonic probe located above or to the side of the surgical bed top surface. The ultrasonic transducer array 260 is mounted to the rotational/translational apparatus 362 (e.g., X-translational stage 562 in FIGS. 5A and 5B, X-translational stage 762 in FIGS. 7A and 7B, an X-Y stage, an articulating arm configured to rotate/translation in 1-6 DOF, etc.) to enable translation/rotation of the ultrasonic transducer array 260 during image acquisition. According to one aspect, the rotational/translational apparatus 362 may be able to rotate and translate with one (1) to six (6) degrees of freedom (DOF).

The energy transmissive surgical bed top apparatus 320 may include one or more additional materials or features without departing from the scope of the disclosure. For example, there may be an acoustic coupling material and a plastic protective film located between the fluid container 330 and the patient 301 during imaging. In one aspect, the fluid container 330, energy transmissive layer 340 and tank 350 have material properties of low acoustic impedance and features that enable transmission of acoustic energy with minimal absorption and low attenuation.

The real-time surgical imaging system 300 also includes one or more pre-amplifiers 382 for boosting (increasing amplitude) acoustic signals communicated from the ultrasonic transducer array 360, one or more data acquisition systems (DAQs) 384 for digitizing the one or more boosted acoustic signals, and a computing device 390 for receiving the digitized acoustic data from the DAQ(s) 384. The computing device 390 also includes one or more processors and/or other circuitry 392 in electrical communication with the DAQ(s) 384 to receive digitized acoustic data. The one or more processors and/or other circuitry 392 execute instructions to perform image processing operations. The computing device 390 also includes a computer readable media 394 and an optional (denoted by dotted line) display 396 in electrical communication with the one or more processors and/or other circuitry 392.

During operation, the ultrasonic transducer array 360 is acoustically coupled to or otherwise in acoustic communication with the patient 301 during signal acquisition to enable receiving acoustic waves and recording acoustic signals. The acoustic coupling is provided through the layers of acoustic medium in the fluid container 330, energy transmissive layer 340, and acoustic medium 352 in the tank 340. The one or more pre-amplifiers 382 are in electrical communication (directly or via other circuitry) with the ultrasonic transducer array 360 to receive one or more acoustic signals. The pre-amplifier(s) 382 can boost one or more photoacoustic signals received and output boosted acoustic signals. The DAQ(s) 384 is configured to process the acoustic signals, for example, digitize and/or record the digitized photoacoustic data. The DAQ(s) 384 may include at least one digitizer to digitize the signals.

The ultrasonic transducer array 360 includes a plurality of N ultrasonic transducers operable to detect acoustic waves in parallel. The N transducer elements in the ultrasonic transducer array 540 may be arranged in, e.g., a circular array such as a full-ring array or a partial ring array, a 1D linear array, or a two-dimensional (2D) array. The rotational/translational apparatus 362 is coupled to the ultrasonic transducer array 360 to be able to translate/rotate the ultrasonic transducer array 360 to one or more positions during operation (e.g., translate in x-direction along the x-axis shown in FIGS. 7A and 7B x-axis shown n FIG. 7A an 7B). For example, the rotational/translational apparatus 362 may be able to translate/rotate the ultrasonic transducer array 360 to two or more different positions (e.g., between x₁, x₂, x₃, x₄, . . . , xₙ along an x-axis in FIG. 7A an 7B or FIG. 5A an 5B) with a step size between positions. In addition, the rotational/translational apparatus 362 may be able to hold the ultrasonic transducer array 360 at each position for a period of time (e.g., about 1 second, about 2 seconds, between 1-10 seconds, or more than 10 seconds) during an image acquisition. For example, the step size may be in a range of 1-10 mm. An example of a commercially-available linear stage is the KR4610D linear stage made by THK America, Inc.

The one or more DAQ(s) 384 record acoustic signals at time intervals defined by a sampling frequency. In one example, the sampling frequency is in a range from about 4 MHz to about 100-Hz. In another example, the sampling frequency is 40 MHz. According to one aspect, the one or more DAQs 384 and one or more pre-amplifiers 382 provide one-to-one mapped associations with the transducers in the ultrasonic transducer array 360. These one-to-one mapped associations allow for fully parallelized data acquisition of all ultrasonic transducer channels and avoids the need for multiplexing after each energy wave delivery. The plurality of pre-amplifier channels may be directly coupled to the corresponding plurality of ultrasonic transducers or may be coupled with electrical connecting cables. In one aspect, wireless communication may be employed.

The real-time surgical imaging system 300 also includes a computing device 390 having one or more processors or other circuitry 392, an optional (denoted by dashed line) display 396 in electrical communication with the processor(s) 392, and a computer readable media (CRM) 394 in electronic communication with the processor(s) or other circuitry 392. The computer readable media (CRM) 394 may be, e.g., a non-transitory computer readable media. Optionally (denoted by dashed line), the computing device 390 is in electronic communication with the first energy delivery system(s) 370 and/or the second energy delivery system(s) 310 send control signals. The computing device 390 is in electrical communication with the one or more DAQs 384 to receive data transmissions and/or to send control signal(s). The computing device 390 may also be in electronic communication with the pre-amplifier(s) 550 to send control signal(s), e.g., to adjust amplification. The electrical communication between components of the real-time surgical imaging system 300 may be in wired and/or wireless form. One or more of the electrical communications between components of the real-time surgical imaging system 300 may be able to provide power in addition to communicate signals. The computing device 390 may be, for example, a personal computer, an embedded computer, a single board computer (e.g. Raspberry Pi or similar), a portable computation device (e.g. tablet), a controller, or any other computation device or system of devices capable of performing the functions described herein. The computing device 390 may be in electronic communication with the translational/rotational apparatus 362 to send control signals to control the translation/rotation and/or hold positions of the ultrasonic transducer array 360. The processor(s) and/or other circuitry 392 are in electrical communication with the CRM 394 to store and/or retrieve data such as the acoustic data. The processor(s) and/or other circuitry 392 are in electrical communication with the optional display 396 to display data. Although not shown, the computing device 390 may also include a user input component for receiving data from a user.

The one or more processors and/or other circuitry 392 execute instructions stored on the CRM 394 to perform one or more operations of real-time surgical imaging methods. In certain implementations, the processor(s) and/or other circuitry 392 execute instructions to perform one or more of: 1) communicating control signals to one or more components of the system 300, and 2) performing one or more reconstruction operations to reconstruct a two-dimensional or three-dimensional images of the region of interest of the patient 301 using energy data detected by the detection system. For example, the processor(s) and/or other circuitry 392 and/or one or more external processors may execute instructions that communicate control signals to the translational/rotational apparatus 362 to scan the ultrasonic transducer array 360 along one or more axis and/or scan in a rotation about one or more axes and send control signals to the digitizer in the DAQ(s) 384 to simultaneously record acoustic signals received by ultrasonic transducer array 360 from the region of interest.

In some implementations, the real-time surgical imaging system 300 includes one or more communication interfaces (e.g., a universal serial bus (USB) interface). Communication interfaces can be used, for example, to connect various peripherals and input/output (I/O) devices such as a wired keyboard or mouse or to connect a dongle for use in wirelessly connecting various wireless-enabled peripherals. Such additional interfaces also can include serial interfaces such as, for example, an interface to connect to a ribbon cable. It should also be appreciated that the various system components can be electrically coupled to communicate with various components over one or more of a variety of suitable interfaces and cables such as, for example, USB interfaces and cables, ribbon cables, Ethernet cables, among other suitable interfaces and cables.

In various embodiments, an energy transmissive surgical bed top apparatus includes a fluid bag (e.g., fluid bag 530 in FIGS. 5A and 5B). The outer wall of the fluid bag may be made of a soft, flexible, sterile, material transmissive to ultrasound and RF/microwave energy, ensuring minimal attenuation and efficient transmission of energy. Some examples of materials that may be used include a thin polyethylene (PE) film, a polyurethane (PU) film (e.g. medical-grade TPU), a polyvinyl chloride (PVC) film which is soft, plasticized, a silicone elastomer sheet, a mylar (e.g., biaxially oriented PET) film if flexibility is acceptable, a low-density polyethylene (LDPE) or ethylene-vinyl acetate (EVA) film for higher softness. During image acquisition, a thin layer of water or acoustic coupling gel may be applied between the fluid bag and the patient's region of interest.

FIG. 5A is a drawing of an isometric view of components of an example of an energy transmissive surgical bed top apparatus 520, according to certain implementations. FIG. 5B is a cross-sectional view of the energy transmissive surgical bed top apparatus 520 in FIG. 5A. The energy transmissive surgical bed top apparatus 520 may be configured to be coupled to a support assembly of a surgical table or may be coupled to an existing surgical bed top that is coupled to the support assembly.

The energy transmissive surgical bed top apparatus 520 includes a fluid bag 530, an energy transmissive layer 540, a tank 550, an ultrasonic transducer array 560, and an x-translational stage 562. The energy transmissive layer 540 may have material properties that closely match the acoustic impedance of water or acoustic gel to minimize reflection and artifacts. In one implementation, the energy transmissive layer is a layer of polymethylpentene (PMP). A commercially-available example of PMP is TPX® PMP sold by Mitsui Chemicals. In another implementation, the energy transmissive layer is a plastic. Various thicknesses of the energy transmissive layer may be used.

During operation, the tank 550 includes a coupling medium 552 such as water, mineral oil, or other liquid with properties for low absorption of light, sound, and RF/microwave energy. The tank 550 includes a curved surface 551, two flange surfaces 553 that are substantially parallel to a plane at the bed top surface 522, and a lower tank surface 554 substantially parallel to a second surface 524 of the outer portion 521. In another implementation, the second surface 554 of the tank 550 may be inset from the second surface 524 of the outer portion 521. A portion of the energy transmissive layer 540 is coupled to the curved surface 551 and flange surfaces 553 of the tank 550. The energy transmissive layer 540 includes edge portions 541 that overhang onto the bed top surface 522. The fluid bag 530 is coupled to the energy transmissive layer 540 and includes edge portions 531 that overhang onto the edge portions 541 of the energy transmissive layer 540. The tank 550 is located within an aperture 523 formed within an outer bed top portion 521. In this example, the edge portions 531 of the fluid bag 530 and the edge portions 541 of the energy transmissive layer 540 are located above the surgical bed top surface 522.

The transmissive surgical bed top apparatus 520 includes an ultrasonic transducer array 560 (e.g., 1D or 2D ultrasonic transducer array) for detecting acoustic waves and/or transmitting acoustic waves. The ultrasonic transducer array 560 is mounted to the x-translational stage 562 to enable translation (depicted by two double sided arrows) along an x-axis during image acquisition. In other implementations, another rotational/translational apparatus may be used for movement with additional degrees of freedom. For example, a 2D translational stage that stacks two 1D translational stages (e.g., x-translational stage) can be used. As another example, an articulating arm can be used.

FIG. 6 is a drawing of an isometric view of a cutaway portion of components of an example of an energy transmissive surgical bed top apparatus 620, according to certain implementations. The energy transmissive surgical bed top apparatus 620 may be configured to be coupled to a support assembly of a surgical table or may be coupled to an existing surgical bed top that is coupled to the support assembly.

The energy transmissive surgical bed top apparatus 620 includes a fluid bag 630, an energy transmissive layer 640, a tank 650, an ultrasonic transducer array 660, and an x-translational stage 662. The energy transmissive layer 640 may have material properties that closely match the acoustic impedance of water or acoustic gel to minimize reflection and artifacts. In one implementation, the energy transmissive layer is a layer of polymethylpentene (PMP) (e.g., PMP is TPX® PMP sold by Mitsui Chemicals). In another implementation, the energy transmissive layer is a plastic. Various thicknesses of the energy transmissive layer may be used.

During operation, the tank 650 includes a coupling medium 652 such as water, mineral oil, or other liquid with properties for low absorption of light, sound, and RF/microwave energy. The tank 650 includes a curved surface 651, two flange surfaces 653 that are substantially parallel to a plane at the bed top surface 622, and a lower tank surface 654 substantially parallel to a second surface 624 of the outer portion 621. In another implementation, the second surface 654 of the tank 650 may be inset from the second surface 624 of the outer portion 621. A portion of the energy transmissive layer 640 is coupled to the curved surface 651 and flange surfaces 653 of the tank 650. The energy transmissive layer 640 includes edge portions 641 that overhang onto the bed top surface 622. The fluid bag 630 is coupled to the energy transmissive layer 640 and includes edge portions 631 that overhang onto the edge portions 641 of the energy transmissive layer 640. The tank 650 is located within an aperture 623 formed within an outer bed top portion 621. In this example, the edge portions 631 of the fluid bag 630 and the edge portions 641 of the energy transmissive layer 640 are located above the surgical bed top surface 622.

The transmissive surgical bed top apparatus 620 includes an ultrasonic transducer array 660 (e.g., 1D or 2D ultrasonic transducer array) for detecting acoustic waves and/or transmitting acoustic waves. The ultrasonic transducer array 660 is mounted to the x-translational stage 662 to enable translation (depicted by two double sided arrows) along an x-axis during image acquisition. In other implementations, another rotational/translational apparatus may be used for movement with additional degrees of freedom.

FIG. 7A is a drawing of an isometric view of components of an example of an energy transmissive surgical bed top apparatus 720, according to certain implementations. FIG. 7B is a cross-sectional view of the energy transmissive surgical bed top apparatus 720 in FIG. 7A. The energy transmissive surgical bed top apparatus 720 may be configured to be coupled to a support assembly of a surgical table or may be coupled to an existing surgical bed top that is coupled to the support assembly.

The energy transmissive surgical bed top apparatus 720 includes an energy transmissive layer 740, a first tank 750 formed in upper surface of the energy transmissive layer 740, a second tank 730, an ultrasonic transducer array 760, and an x-translational stage 762. The energy transmissive layer 740 may have material properties that closely match the acoustic impedance of water or acoustic gel to minimize reflection and artifacts. In one implementation, the energy transmissive layer is a layer of polymethylpentene (PMP) (e.g., TPX® PMP sold by Mitsui Chemicals). In another implementation, the energy transmissive layer is a plastic. Various thicknesses of the energy transmissive layer may be used.

During operation, the first tank 750 has a coupling medium 752 such as water, mineral oil, or other liquid with properties for low absorption of light, sound, and RF/microwave energy. The first tank 750 includes a curved surface 751, two flange surfaces 753 that are substantially parallel to a plane at the bed top surface 722, and a lower tank surface 754 substantially parallel to a second surface 724 of the outer portion 721. In another implementation, the second surface 754 of the first tank 750 may be inset from the second surface 724 of the outer portion 721. A portion of the energy transmissive layer 740 is coupled to the curved surface 751 and flange surfaces 753 of the first tank 750. The energy transmissive layer 740 includes edge portions 741 that overhang onto the bed top surface 722. In this example, the second tank 730 is formed in the central portion of the energy transmissive layer 740. In another implementation, the second tank 730 may be a separate component coupled to the energy transmissive layer 740. The first tank 750 is located within an aperture 723 formed within an outer bed top portion 721. In this example, the edge portions 741 of the energy transmissive layer 740 are located above the surgical bed top surface 722.

The transmissive surgical bed top apparatus 720 includes an ultrasonic transducer array 760 (e.g., 1D or 2D ultrasonic transducer array) for detecting acoustic waves and/or transmitting acoustic waves. The ultrasonic transducer array 760 is mounted to the x-translational stage 762 to enable translation (depicted by two double sided arrows) along an x-axis during image acquisition. In other implementations, another rotational/translational apparatus may be used for movement with additional degrees of freedom. For example, a 2D translational stage that stacks two 1D translational stages (e.g., x-translational stage) can be used. As another example, an articulating arm can be used. FIG. 8 is a drawing depicting a real-time surgical imaging system 800 including an energy transmissive surgical bed top apparatus 820, according to various implementations. The elements of the energy transmissive surgical bed top apparatus 820 are analogous to the elements of the energy transmissive surgical bed top apparatus 520 shown in FIGS. 5A and 5B. For the sake of brevity, the prior discussion of the elements of the energy transmissive surgical bed top apparatus 820 may be assumed to be equally applicable, unless indicated otherwise in the following discussion, to the analogous counterparts of those of the energy transmissive surgical bed top apparatus 520 shown in FIGS. 5A and 5B that share the same last two digits in their respective callouts as in FIG. 8.

In the illustrated example, energy transmissive surgical bed top apparatus 820 is coupled to a support assembly 810 of a surgical table 804. In another implementation, the energy transmissive surgical bed top apparatus 820 may be coupled to an existing surgical bed top that is coupled to the support assembly. The illustration is shown at an instant in time during an imaging operation when a patient 801 is disposed on top of the energy transmissive surgical bed top apparatus 820. At other times, the patient 801 may not be disposed on the energy transmissive surgical bed top apparatus 820.

The energy transmissive surgical bed top apparatus 820 includes a fluid bag 830, an energy transmissive layer, a tank with a coupling medium, an ultrasonic transducer array 860, and an x-translational stage 862. The ultrasonic transducer array 860 is mounted to the x-translational stage 862. The fluid bag 830 is coupled to the energy transmissive layer and includes edge portions that overhang onto bed top surface 822. The tank is located within an aperture 823 in the bed top. The tank includes a coupling medium such as water, mineral oil, or other liquid with properties for low absorption of light, sound, and RF/microwave energy.

In various embodiments, real-time surgical imaging systems provide a surgical platform that can implement multiple imaging modalities and/or energy-implemented surgical techniques with free-space and guided energy delivery systems. For example, a microwave antenna can be used to deliver RF/microwave energy to excite tissues inducing thermoacoustic waves and also can be used to focus energy on a targeted area to enable localize heating and ablation for cauterizing and cutting. As another example, an ultrasonic probe can be used for transmission of ultrasound waves for use in ultrasound tomography for detailed imaging. As another example, an ultrasonic probe can be used for transmission of ultrasound waves for use in ultrasound tomography for detailed imaging. As another example, one or more optical fibers can be used to deliver light directly to a surgical site for therapeutic or imaging purposes imaging.

By way of an illustrated example, the real-time surgical imaging system 800 in FIG. 8 includes energy delivery systems including an ultrasonic probe 812 for transmitting/receiving acoustic waves, one or more optical fibers 813 (e.g., in the form of a fiber bundle), and a RF/microwave antenna 815. In one aspect, the ultrasonic probe 812, the one or more optical fibers 813, and/or the RF/microwave antenna 815 may be mounted to articulating arms. The real-time surgical imaging system 800 also includes a computing system 890 in electronic communication with the ultrasonic transducer array and the ultrasonic probe 812. The computing device 890 includes one or more processors or other circuitry, a computer readable media, and an optional display. The real-time surgical imaging system 800 may include one or more additional, fewer, or alternative elements without departing from the scope of the disclosure.

At the instant time shown in the illustrated example, the RF/microwave antenna 814 is delivering modulated radio frequency signals into the tissues of the patient 801 exciting a RF/microwave excited volume 816, leading to leading to absorption-specific thermoplastic expansion released as thermoacoustic waves. The ultrasonic transducer array and/or the ultrasonic probe 812 can detect the thermoacoustic waves. The computing device 890 can execute instructions to perform a thermoacoustic tomography (TAT) image reconstruction procedure (e.g., inverse reconstruction algorithm) to generate thermoacoustic images of a region of interest in the RF/microwave excited volume 816. Also occurring at the instant in time, the one or more optical fibers 813 deliver near-infrared (NIR) wavelengths to the tissues exciting an optically excited volume 814 which induces photoacoustic waves. The ultrasonic transducer array and/or the ultrasonic probe 812 can detect the photoacoustic waves and the computing device 890 can execute instructions to perform a photoacoustic tomography (PAT) reconstruction procedure (e.g., procedure including a forward-model-based iterative method, a time-reversal method, or a universal back-projection (UBP) method) to reconstruct photoacoustic images a region of interest in the optically excited volume 814. The ultrasonic transducer array and/or the ultrasonic probe 812 can also detect the ultrasound waves delivered by the ultrasonic transducer array and/or the ultrasonic probe 812 and reflected by the tissues. The ultrasound signals can be used to reconstruct ultrasound images.

FIG. 9 is a drawing depicting the real-time surgical imaging system 800 in FIG. 8 with the ultrasonic probe 812, one or more optical fibers 813, RF/microwave antenna 815 in second positions to take images of a different region of interest.

### Surgical Robots

Many standard imaging systems are not compatible with robotic systems. For example, traditional MRI imaging is not compatible with the metallic components of most surgical robots due to the strong magnetic fields generated. Also, since surgical robotics require real-time imaging for guidance, standard modalities like CT, PET, and SPECT are not capable of providing immediate feedback due to their long acquisition and processing times. Moreover, the spatial resolution of modalities like PET and SPECT may not be sufficient for the fine control required in robotic surgery, where millimeter or sub-millimeter precision is often necessary.

In various implementations disclosed herein, real-time surgical imaging systems provide real-time feedback and high-resolution imaging which enables compatibility with surgical robots. In various examples, real-time surgical imaging systems integrate one or more surgical robots (sometimes referred to herein as “robotic systems”) to enable enhance precision, flexibility, and control during procedures.

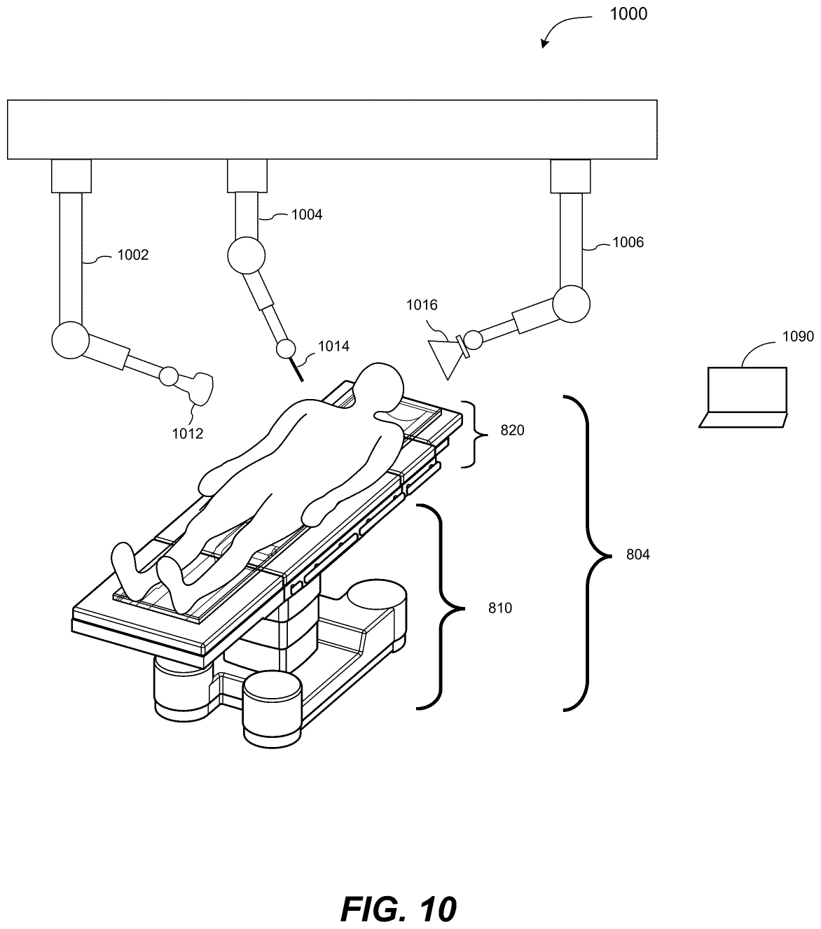

FIG. 10 is a drawing depicting a real-time surgical imaging system 1000 with a robotic system 1010, according to an embodiment. Real-time surgical imaging system 1000 also includes the surgical table 1004 shown in FIGS. 8 and 9. In the illustrated example, energy transmissive surgical bed top apparatus 1020 is coupled to a support assembly 1010 of a surgical table 1004. In another implementation, the energy transmissive surgical bed top apparatus 1020 may be coupled to an existing surgical bed top that is coupled to the support assembly. The illustration is shown at an instant in time during an imaging operation when a patient 1001 is disposed on top of the energy transmissive surgical bed top apparatus 1020. At other times, the patient 1001 may not be disposed on the energy transmissive surgical bed top apparatus 1020.

The robotic system 1010 includes a first robotic arm 1002 coupled to an ultrasonic probe, a second robotic arm 1004 coupled to one or more optical fibers 1014, and a third robotic arm 1006 coupled to a microwave antenna 1016. The real-time surgical imaging system 1000 also includes a computing system 1090 in electronic communication with the ultrasonic transducer array and the ultrasonic probe 1012. The computing device 1090 includes one or more processors or other circuitry, a computer readable media, and an optional display. The RF/microwave antenna 1014 can deliver modulated radio frequency signals into the tissues of the patient 1001 exciting the tissues, leading to leading to absorption-specific thermoclastic expansion released as thermoacoustic waves. The ultrasonic transducer array and/or the ultrasonic probe 1012 can detect the thermoacoustic waves and the computing device 1090 can execute instructions to perform a thermoacoustic tomography (TAT) reconstruction procedure (e.g., inverse reconstruction algorithm) to generate thermoacoustic images of a region of interest. The one or more optical fibers 1013 can deliver near-infrared (NIR) wavelengths to the tissues inducing photoacoustic waves. The ultrasonic transducer array and/or the ultrasonic probe 1012 can detect the photoacoustic waves and the computing device 1090 can execute instructions to perform a photoacoustic tomography (PAT) reconstruction procedure (e.g., procedure including a forward-model-based iterative method, a time-reversal method, or a universal back-projection (UBP) method) to reconstruct photoacoustic images of a region of interest. Also, the ultrasonic transducer array and/or the ultrasonic probe 1012 can detect ultrasound waves delivered by the ultrasonic transducer array and/or the ultrasonic probe 1012 and reflected by the tissues. The ultrasound signals can be used to reconstruct ultrasound images. The real-time surgical imaging system 1000 may include one or more additional, fewer, or alternative elements without departing from the scope of the disclosure.

### IV. Examples of real-time surgical imaging methods

FIG. 11 depicts a flowchart 1100 of a real-time surgical imaging method, according to embodiments. In some cases, the method may be implemented by a computing device of a real-time surgical imaging system (e.g., computing device 190 in FIG. 1, computing device 390 in FIG. 3, computing device 890 in FIG. 8, computing device 890 in FIG. 9, or computing device 1090 in FIG. 10), according to various implementations. In some cases, one or more of the operations are performed in real-time.

At operation 1110, digitized energy signal data obtained from memory or from data acquisiton system(s). The energy signals are associated with energy delivered by components of one or more energy deliver systems (e.g, microwave antenna/waveguide, ultrasonic probe, ultrasonic transducer array, one or more optical fibers, etc.). For example, digitized acoustic signals may be received from a data acquisition system(s) (e.g., data acquisition system(s) 384 in FIG. 3). The acoustic signals may be detected by an ultrasonic ultrasonic transducer array beneath the surgical bed top surface or an ultrasonic probe above or to the side of the surgical bed top. In one implementation, the acoustic signals may be induced by near infrared light delivered by one or more optical fibers from above the surgical field. In another implementation, the acoustic signals may be induced by RF/microwave energy delivered by an RF/microwave antenna or waveguide from above the surgical field. In another implementation, the acoustic signals may result from detection of ultrasound waves reflected from tissues where the ultrasonic waves are delivered by an ultrasonic ultrasonic transducer array beneath the surgical bed top surface or an ultrasonic probe above or to the side of the surgical bed top.

At operation 1120, reconstruction techniques are used to reconstruct one or more images of a region of interest from the digitized energy signal data. Image reconstruction may include (i) reconstructing a plurality of two-dimensional (2D) images based on different scanning locations of an ultrasonic transducer array or ultrasonic probe and/or (ii) generating a volumetric three-dimensional image by combining the two-dimensional images for the volume scanned by the ultrasonic sensor array or ultrasonic probe. The three-dimensional (3D) image data may be used to co-register to pre-operative 3D images.

In some cases, a computing device can execute instructions to perform a thermoacoustic tomography (TAT) image reconstruction procedure such as an inverse reconstruction algorithm to generate one or more thermoacoustic images. Some examples of inverse reconstruction methods that can be used include: (i) forward-model-based iterative methods, (ii) time-reversal methods, and (iii) back projection methods. A 3D back projection method can be used to reconstruct a 3D volumetric image and a 2D back projection method can be used to reconstruct a 2D image. An example of a back projection method is the universal back-projection process described in U.S. patent application Ser. No. 17/090,752, titled “SPATIOTEMPORAL ANTIALIASING IN PHOTOACOUSTIC COMPUTED TOMOGRAPHY” and filed on Nov. 5, 2020, which is hereby incorporated by reference for this description. Another example of a back-projection method can be found in Anastasio, M. A. et al., “Half-time image reconstruction in thermoacoustic tomography,” IEEE Trans., Med. Imaging 24, pp 199-210 (2005). In another aspect, a dual-speed-of sound (dual-SOS) photoacoustic reconstruction process may be used. An example of a single-impulse panoramic photoacoustic computed tomography system that employs a dual-SOS photoacoustic reconstruction process is described in U.S patent application 2019/0307334, titled “SINGLE-IMPULSE PANORAMIC PHOTOACOUSTIC COMPUTED TOMOGRAPHY” and filed on May 29, 2019. In one case, a computing device can execute instructions to perform a photoacoustic tomography (PAT) reconstruction procedure (e.g., procedure including a forward-model-based iterative method, a time-reversal method, or a universal back-projection (UBP) method) to reconstruct photoacoustic images. In one case, a computing device can execute instructions to perform an ultrasound reconstruction method to generate one or more ultrasound images.

At operation 1130, the one or more reconstructed images are co-registered with corresponding one or more pre-operative images. Co-registration aligns pre-surgical data with the patient's current anatomy, enabling highly accurate surgical guidance. This process compensates for patient movement, anatomical shifts, or tissue deformation, ensuring that surgical navigation reflects real-time conditions. By integrating imaging and anatomical data seamlessly, it enhances precision in targeting, reduces the risk of damage to critical structures, and can shorten operative time.

At optional (denoted by dashed line) operation 1140, one or more visualizations of the co-registered data are displayed to user interface. For example, a computing device may transmit image data to a user interface on a display.

FIG. 12 depicts a flowchart 1200 of a real-time surgical imaging method, according to embodiments. In some cases, the method may be implemented by a computing device of a real-time surgical imaging system (e.g., computing device 190 in FIG. 1, computing device 390 in FIG. 3, computing device 890 in FIG. 8, computing device 890 in FIG. 9, or computing device 1090 in FIG. 10). In various implementations, one or more of the operations are performed in real-time.

At operation 1210, one or more energy delivery systems (e.g, microwave antenna/waveguide, ultrasonic probe, ultrasonic transducer array, one or more optical fibers, etc.) deliver energy to a region of interest of a patient. For example, a computing device may send control signals to the energy delivery systems to cause delivery of energy. As another example, the one or more energy delivery systems may be programmed to deliver energy. In some cases, the energy delivery from multiple energy delivery systems may be interleaved (e.g., ultrasound pulses interleaved with RF waves). The energy delivery system(s) may deliver energy from beneath the bed top surface, from the side of the bed top, and/or from above bed top surface. In one case, a laser source may be triggered to cause emission of near infrared light. The laser source may be optically coupled to one or more optical fibers that deliver the infrared light to a region of interest. In some instances, the computing device may send control systems to a robotic system that is coupled to the one or more optical fibers to maneuver the optical fiber(s) to the region of interest. In another case, an RF/microwave source may be triggered to cause emission of RF/microwave energy. The RF/microwave source may be in electronic communication with a RF/microwave antenna or waveguide that can deliver the RF/microwave energy to a region of interest. In some instances, the computing device may send control systems to a robotic system that is coupled to the RF/microwave antenna or waveguide to maneuver it to the region of interest.

At operation 1120, the ultrasonic transducer array and/or ultrasonic probe is scanned (translated and/or rotated) to different positions during acquisition of acoustic signals. For example, the ultrasonic transducer array or ultrasonic probe may be scanned to multiple x-direction locations (e.g., different locations x₁, x₂, . . . along an x-axis of a linear stage) and held in each position for a period of time during which DAQ(s) record data. Some examples of time periods that may be used include: about 10 seconds, about 15 seconds, and about 20 seconds. In another example, the time period is in a range of about 10 seconds to about 20 seconds. At each position, acoustic data is continuously recorded at a certain sampling rate to monitor the cross section. The 2-D image data may be combined to generate three-dimensional (3D) image data for co-registration with 3D preoperative images.

At operation 1130, reconstruction techniques are used to reconstruct one or more images of a region of interest from digitized acoustic signals. Image reconstruction may include (i) reconstructing a plurality of two-dimensional (2D) images based on different scanning locations of an ultrasonic transducer array or ultrasonic probe and/or (ii) generating a volumetric three-dimensional image by combining the two-dimensional images for the volume scanned by the ultrasonic sensor array or ultrasonic probe. The three-dimensional (3D) image data may be used to co-register to pre-operative 3D images.

At optional (denoted by dashed line) operation 1140, one or more acoustic images are displayed to user interface. For example, a computing device may transmit image data to a user interface on a display.

### IV. Computational Systems

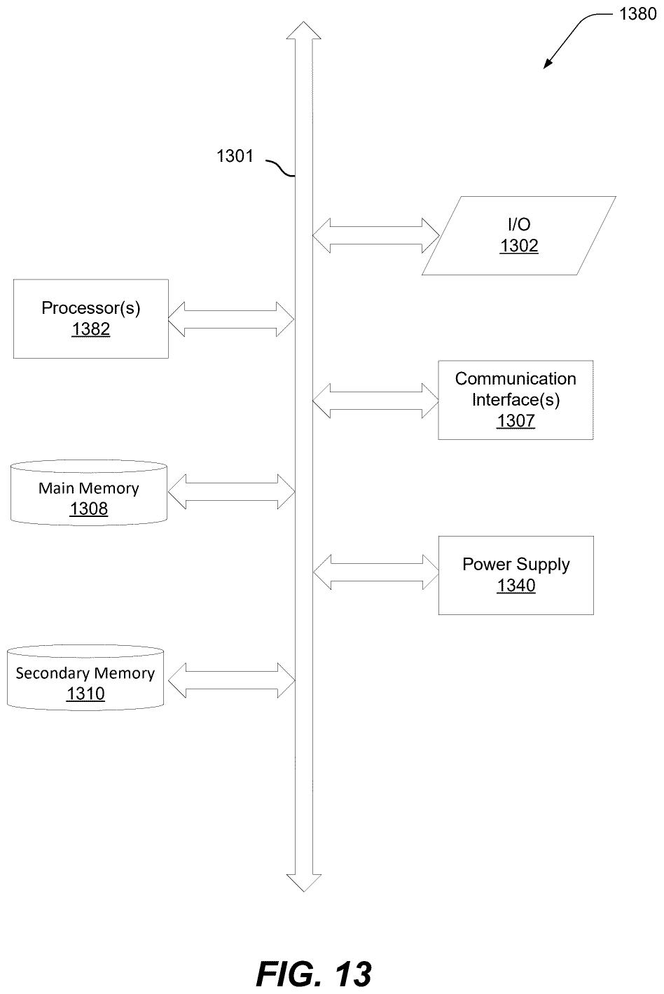

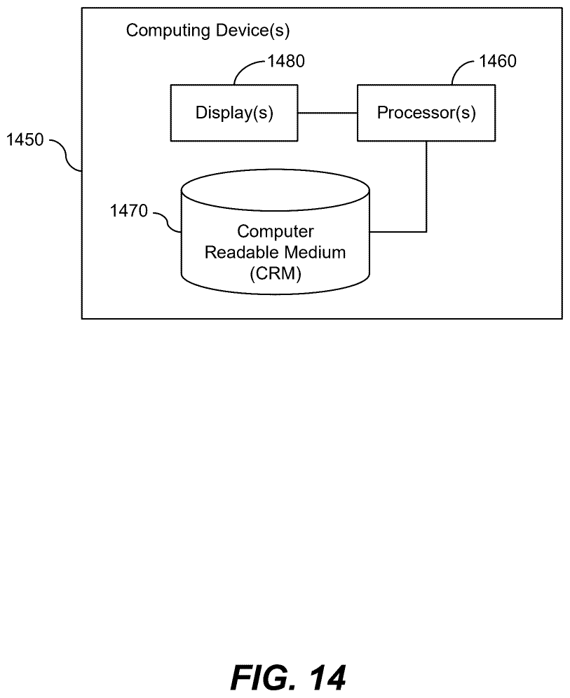

The techniques described above may be implemented using one or more computing devices. FIGS. 13 and 14 illustrate examples of computing devices that may be used, e.g., to implement functions of real-time surgical imaging methods described herein.

In FIG. 13, the computing device(s) 1380 includes a bus 1301 coupled to an input/output (I/O) subsystem 1302, one or more processors 1382, one or more communication interfaces 1307, a main memory 1308, a secondary memory 1310, and a power supply 1340. One of more of these components may be in separate housing. According to certain implementations, the illustrated components may be examples of components that are part of computing device 190 in FIG. 1, computing device 1390 in FIG. 3, computing device 890 in FIGS. 8 and 9, or computing device 1090 in FIG. 10. Computing device 1380 includes I/O subsystem 1302, which includes, or is in communication with, one or more components which may implement an interface for interacting with human users and/or other computer devices depending upon the application.

Certain embodiments disclosed herein may be implemented in program code on computing device 1380 with I/O subsystem 1302 used to receive input program statements and/or data from a human user (e.g., via a graphical user interface (GUI), a keyboard, touchpad, etc.) and to display them back to the user, for example, on a display. The I/O subsystem 1302 may include, e.g., a keyboard, mouse, graphical user interface, touchscreen, or other interfaces for input, and, e.g., an LED or other flat screen display, or other interfaces for output. Other elements of embodiments may be implemented with a computer system like that of computer system 1300 without I/O subsystem 1302. According to various embodiments, a processor may include a CPU, GPU or computer, analog and/or digital input/output connections, controller boards, etc.

Program code may be stored in non-transitory computer readable media such as secondary memory 1310 or main memory 1308 or both. One or more processors 1304 may read program code from one or more non-transitory media and execute the code to enable computing device 1380 to accomplish the methods performed by various embodiments described herein, such as APIC imaging methods. Those skilled in the art will understand that the one or more processors 1382 may accept source code and interpret or compile the source code into machine code that is understandable at the hardware gate level of the one or more processors 1382.

Communication interfaces 1307 may include any suitable components or circuitry used for communication using any suitable communication network (e.g., the Internet, an intranet, a wide-area network (WAN), a local-area network (LAN), a wireless network, a virtual private network (VPN), and/or any other suitable type of communication network). For example, communication interfaces 1307 can include network interface card circuitry, wireless communication circuitry, etc.

In certain embodiments, computing device 1380 may be part of or connected to a controller that is employed to control functions of various system components described herein. For example, computing device 1380 may control recording of signals by DAQ(s) or data acquisition by an ultrasonic transducer array or ultrasonic probe and/or delivery of energy an energy source. The controller will typically include one or more memory devices and one or more processors. The processor may include a CPU or computer, analog and/or digital input/output connections, stepper motor controller boards, etc.

In FIG. 14, the computing device(s) 1450 includes one or more processors 1460 (e.g., microprocessors), a non-transitory computer readable medium (CRM) 1470 in communication with the processor(s) 1460, and one or more displays 1480 also in communication with processor(s) 1460. Processor(s) 1460 is in electronic communication with CRM 1470 (e.g., memory). Processor(s) 1460 is also in electronic communication with display(s) 1480, e.g., to display image data, text, etc. on display 1480. Processor(s) 1460 may retrieve and execute instructions stored on the CRM 1470 to perform one or more functions described above. For example, processor(s) 1460 may execute instructions to perform one or more operations to generate an outcome prediction, train a DNN to generate outcome predictions, etc. The CRM (e.g., memory) 1470 can store instructions for performing one or more functions of the described above. These instructions may be executable by processor(s) 1470. CRM 1470 can also store raw images, e.g., microscopy images, sub-images sampled from an image, or the like.

Many types of computing devices having any of various computer architectures may be employed as the disclosed systems for implementing algorithms. For example, the computing devices may include software components executing on one or more general purpose processors or specially designed processors such as Application Specific Integrated Circuits (ASICs) or programmable logic devices (e.g., Field Programmable Gate Arrays (FPGAs)). Further, the systems may be implemented on a single device or distributed across multiple devices. The functions of the computational elements may be merged into one another or further split into multiple sub-modules.

At one level a software element is implemented as a set of commands prepared by the programmer/developer. However, the module software that can be executed by the computer hardware is executable code committed to memory using “machine codes” selected from the specific machine language instruction set, or “native instructions,” designed into the hardware processor. The machine language instruction set, or native instruction set, is known to, and essentially built into, the hardware processor(s). This is the “language” by which the system and application software communicates with the hardware processors. Each native instruction is a discrete code that is recognized by the processing architecture and that can specify particular registers for arithmetic, addressing, or control functions; particular memory locations or offsets; and particular addressing modes used to interpret operands. More complex operations are built up by combining these simple native instructions, which are executed sequentially, or as otherwise directed by control flow instructions.

The inter-relationship between the executable software instructions and the hardware processor is structural. In other words, the instructions per se are a series of symbols or numeric values. They do not intrinsically convey any information. It is the processor, which by design was preconfigured to interpret the symbols/numeric values, which imparts meaning to the instructions.

The algorithms used herein may be configured to execute on a single machine at a single location, on multiple machines at a single location, or on multiple machines at multiple locations. When multiple machines are employed, the individual machines may be tailored for their particular tasks. For example, operations requiring large blocks of code and/or significant processing capacity may be implemented on large and/or stationary machines.

In addition, certain embodiments relate to tangible and/or non-transitory computer readable media or computer program products that include program instructions and/or data (including data structures) for performing various computer-implemented operations. Examples of computer-readable media include, but are not limited to, memory devices, phase-change devices, magnetic media such as disk drives, magnetic tape, optical media such as CDs, magneto-optical media, and hardware devices that are specially configured to store and perform program instructions, such as read-only memory devices (ROM) and random access memory (RAM). The computer readable media may be directly controlled by an end user or the media may be indirectly controlled by the end user. Examples of directly controlled media include the media located at a user facility and/or media that are not shared with other entities. Examples of indirectly controlled media include media that is indirectly accessible to the user via an external network and/or via a service providing shared resources such as the “cloud.” Examples of program instructions include both machine code, such as produced by a compiler, and files containing higher level code that may be executed by the computer using an interpreter.

In some embodiments, code executed during generation or execution of various models on an appropriately programmed system can be embodied in the form of software elements which can be stored in a nonvolatile storage medium (such as optical disk, flash storage device, mobile hard disk, etc.), including a number of instructions for making a computing device (such as personal computers, servers, network equipment, etc.). In various embodiments, the data or information employed in the disclosed methods and apparatus is provided in an electronic format. Such data or information may include design layouts, fixed parameter values, floated parameter values, feature profiles, metrology results, and the like. As used herein, data or other information provided in electronic format is available for storage on a machine and transmission between machines. Conventionally, data in electronic format is provided digitally and may be stored as bits and/or bytes in various data structures, lists, databases, etc. The data may be embodied electronically, optically, etc.

### Example Embodiments

Embodiment 1: A real-time surgical imaging system, comprising: a bed top surface configured to receive at least a portion of a patient; an energy transmissive layer; and one or more first energy delivery systems configured to deliver energy from below the bed top surface through the energy transmissive layer to a region of interest in the patient; and a detection system for detecting energy received from the region of interest.
Embodiment 2: The real-time surgical imaging system of embodiment 1, further comprising: a fluid container configured to acoustic couple to at least a portion of the patient; and a tank of coupling medium, wherein the energy transmissive layer is located between the fluid container and the tank of coupling medium.
Embodiment 3: The real-time surgical imaging system of embodiment 1, further comprising one or more second energy delivery systems for delivering energy from above the bed top surface to the region of interest.
Embodiment 4: The real-time surgical imaging system of embodiment 3, further comprising one or more robotic devices configured to rotate and/or translate the one or more second energy delivery systems.
Embodiment 5: The real-time surgical imaging system of embodiment 3, wherein the detection system comprises one or more detector devices located above the bed top surface.
Embodiment 6: The real-time surgical imaging system of embodiment 3, wherein the one or more second energy delivery systems comprise a light source and/or one or more antennas or waveguides for transmitting radio frequency energy.
Embodiment 7: The real-time surgical imaging system of embodiment 1, wherein the one or more first energy delivery systems comprise one or more of (i) one or more ultrasonic transducers, (ii) one or more antennas or waveguides for transmitting radio frequency energy, and (iii) a light source.
Embodiment 8: The real-time surgical imaging system of embodiment 1, wherein the detection system comprises: an ultrasonic transducer array configured to detect the energy received from the region of interest through the energy transmissive layer; and a mechanism coupled to the ultrasonic transducer array to rotate and/or translate the ultrasonic transducer array during operation.
Embodiment 9: The real-time surgical imaging system of embodiment 8, wherein the ultrasonic transducer array and the mechanism are located below the bed top surface.
Embodiment 10: The real-time surgical imaging system of embodiment 8, further comprising: one or more pre-amplifiers in communication with the ultrasonic transducer array; and one or more data acquisition systems in communication with the one or more pre-amplifier.
Embodiment 11: The real-time surgical imaging system of embodiment 1, wherein acoustic coupling to the patient is through (i) a tank comprising liquid or (ii) a fluid bag.
Embodiment 12: The real-time surgical imaging system of embodiment 1, further comprising a computing system configured to reconstruct one or more images based at least in part on energy signals detected by the detection system.
Embodiment 13: An energy transmissive surgical bed top apparatus, comprising: a fluid container configured to acoustically couple to at least a portion of a patient being imaged; an energy transmissive transparent layer; a tank with a coupling medium, wherein the energy transmissive layer is located between the fluid container and the tank; an ultrasonic transducer array in the tank, the ultrasonic transducer array configured to detect energy received through the energy transmissive layer; and a mechanism coupled to the ultrasonic transducer array to rotate and/or translate the ultrasonic transducer array during operation.
Embodiment 14: The energy transmissive surgical bed top apparatus of embodiment 13, wherein the fluid container is (i) another tank with liquid or (ii) a fluid bag.
Embodiment 15: The energy transmissive surgical bed top apparatus of embodiment 13, wherein the energy transmissive transparent layer comprises polymethylpentene.
Embodiment 16: The energy transmissive surgical bed top apparatus of embodiment 13, wherein the energy transmissive transparent layer is part of the fluid container or part of the tank.
Embodiment 17: The energy transmissive surgical bed top apparatus of embodiment 13, wherein the energy transmissive surgical bed top apparatus is configured to (i) be located on top of an upper surface of an operating table or (ii) replace a bed top portion of an operating table.
Embodiment 18: The energy transmissive surgical bed top apparatus of embodiment 13, wherein the energy transmissive surgical bed top apparatus is configured to couple to a support apparatus of an operating table.
Embodiment 19: The energy transmissive surgical bed top apparatus of embodiment 13, wherein the tank comprises a curved surface with the energy transmissive layer disposed at least partially thereon.
Embodiment 20: The real-time surgical imaging method of embodiment 13, a real-time surgical imaging method, comprising: acquiring first energy signals associated with energy delivered during a surgical operation by one or more first energy delivery systems to a region of interest of a patient from below a bed top surface through an energy transmissive layer; reconstructing one or more first inoperative images of the region of interest based on the energy signals; co-registering the one or more first images with one or more corresponding pre-operative images; and displaying co-registered images during the surgical operation.
Embodiment 21: The real-time surgical imaging method of embodiment 20, further comprising acquiring second energy signals associated with energy delivered during the surgical operation by one or more second energy delivery systems to the region of interest from above the bed top surface.
Embodiment 22: The real-time surgical imaging method of embodiment 20, reconstructing one or more second images of the region of interest based on the energy signals; co-registering the one or more second images with one or more corresponding one or more pre-operative images; and displaying co-registered images.

Modifications, additions, or omissions may be made to any of the above-described embodiments without departing from the scope of the disclosure. Any of the embodiments described above may include more, fewer, or other features without departing from the scope of the disclosure. Additionally, the steps of described features may be performed in any suitable order without departing from the scope of the disclosure. Also, one or more features from any embodiment may be combined with one or more features of any other embodiment without departing from the scope of the disclosure. The components of any embodiment may be integrated or separated according to particular needs without departing from the scope of the disclosure.

It should be understood that certain aspects described above can be implemented in the form of logic using computer software in a modular or integrated manner. Based on the disclosure and teachings provided herein, a person of ordinary skill in the art will know and appreciate other ways and/or methods to implement the present invention using hardware and a combination of hardware and software.

Any of the software components or functions described in this application, may be implemented as software code using any suitable computer language and/or computational software such as, for example, Java, C, C#, C++ or Python, LabVIEW, Mathematica, or other suitable language/computational software, including low level code, including code written for field programmable gate arrays, for example in VHDL. The code may include software libraries for functions like data acquisition and control, motion control, image acquisition and display, etc. Some or all of the code may also run on a personal computer, single board computer, embedded controller, microcontroller, digital signal processor, field programmable gate array and/or any combination thereof or any similar computation device and/or logic device(s). The software code may be stored as a series of instructions, or commands on a CRM such as a random access memory (RAM), a read only memory (ROM), a magnetic media such as a hard-drive or a floppy disk, or an optical media such as a CD-ROM, or solid stage storage such as a solid state hard drive or removable flash memory device or any suitable storage device. Any such CRM may reside on or within a single computational apparatus, and may be present on or within different computational apparatuses within a system or network. Although the foregoing disclosed embodiments have been described in some detail to facilitate understanding, the described embodiments are to be considered illustrative and not limiting. It will be apparent to one of ordinary skill in the art that certain changes and modifications can be practiced within the scope of the appended claims.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and can also cover other unlisted steps. Similarly, any composition or device that “comprises,” “has” or “includes” one or more features is not limited to possessing only those one or more features and can cover other unlisted features.

All methods described herein can be performed in any suitable order unless otherwise indicated herein or otherwise clearly contradicted by context. The use of any and all examples, or exemplary language (e.g., “such as”) provided with respect to certain embodiments herein is intended merely to better illuminate the present disclosure and does not pose a limitation on the scope of the present disclosure otherwise claimed. No language in the specification should be construed as indicating any non-claimed element essential to the practice of the present disclosure.

Groupings of alternative elements or embodiments of the present disclosure disclosed herein are not to be construed as limitations. Each group member can be referred to and claimed individually or in any combination with other members of the group or other elements found herein. One or more members of a group can be included in, or deleted from, a group for reasons of convenience or patentability. When any such inclusion or deletion occurs, the specification is herein deemed to contain the group as modified thus fulfilling the written description of all Markush groups used in the appended claims.

What is claimed is:

1. A real-time surgical imaging system, comprising:
a bed top surface configured to receive at least a portion of a patient;
an energy transmissive layer; and
one or more first energy delivery systems configured to deliver energy from below the bed top surface through the energy transmissive layer to a region of interest in the patient; and
a detection system for detecting energy received from the region of interest.

2. The real-time surgical imaging system of claim 1, further comprising:
a fluid container configured to acoustic couple to at least a portion of the patient; and
a tank of coupling medium,
wherein the energy transmissive layer is located between the fluid container and the tank of coupling medium.

3. The real-time surgical imaging system of claim 1, further comprising one or more second energy delivery systems for delivering energy from above the bed top surface to the region of interest.

4. The real-time surgical imaging system of claim 3, further comprising one or more robotic devices configured to rotate and/or translate the one or more second energy delivery systems.

5. The real-time surgical imaging system of claim 3, wherein the detection system comprises one or more detector devices located above the bed top surface.

6. The real-time surgical imaging system of claim 3, wherein the one or more second energy delivery systems comprise a light source and/or one or more antennas or waveguides for transmitting radio frequency energy.

7. The real-time surgical imaging system of claim 1, wherein the one or more first energy delivery systems comprise one or more of (i) one or more ultrasonic transducers, (ii) one or more antennas or waveguides for transmitting radio frequency energy, and (iii) a light source.

8. The real-time surgical imaging system of claim 1, wherein the detection system comprises:
an ultrasonic transducer array configured to detect the energy received from the region of interest through the energy transmissive layer; and
a mechanism coupled to the ultrasonic transducer array to rotate and/or translate the ultrasonic transducer array during operation.

9. The real-time surgical imaging system of claim 8, wherein the ultrasonic transducer array and the mechanism are located below the bed top surface.

10. The real-time surgical imaging system of claim 8, further comprising:
one or more pre-amplifiers in communication with the ultrasonic transducer array; and
one or more data acquisition systems in communication with the one or more pre-amplifiers.

11. The real-time surgical imaging system of claim 1, wherein acoustic coupling to the patient is through (i) a tank comprising liquid or (ii) a fluid bag.

12. The real-time surgical imaging system of claim 1, further comprising a computing system configured to reconstruct one or more images based at least in part on energy signals detected by the detection system.

13. An energy transmissive surgical bed top apparatus, comprising:
a fluid container configured to acoustically couple to at least a portion of a patient being imaged;
an energy transmissive transparent layer;
a tank with a coupling medium, wherein the energy transmissive layer is located between the fluid container and the tank;
an ultrasonic transducer array in the tank, the ultrasonic transducer array configured to detect energy received through the energy transmissive layer; and
a mechanism coupled to the ultrasonic transducer array to rotate and/or translate the ultrasonic transducer array during operation.

14. The energy transmissive surgical bed top apparatus of claim 13, wherein the fluid container is (i) another tank with liquid or (ii) a fluid bag.

15. The energy transmissive surgical bed top apparatus of claim 13, wherein the energy transmissive transparent layer comprises polymethylpentene.

16. The energy transmissive surgical bed top apparatus of claim 13, wherein the energy transmissive transparent layer is part of the fluid container or part of the tank.

17. The energy transmissive surgical bed top apparatus of claim 13, wherein the energy transmissive surgical bed top apparatus is configured to (i) be located on top of an upper surface of an operating table or (ii) replace a bed top portion of an operating table.

18. The energy transmissive surgical bed top apparatus of claim 13, wherein the energy transmissive surgical bed top apparatus is configured to couple to a support apparatus of an operating table.

19. The energy transmissive surgical bed top apparatus of claim 13, wherein the tank comprises a curved surface with the energy transmissive layer disposed at least partially thereon.

20. A real-time surgical imaging method, comprising:
acquiring first energy signals associated with energy delivered during a surgical operation by one or more first energy delivery systems to a region of interest of a patient from below a bed top surface through an energy transmissive layer;
reconstructing one or more first inoperative images of the region of interest based on the energy signals;
co-registering the one or more first images with one or more corresponding pre-operative images; and
displaying co-registered images during the surgical operation.

21. The real-time surgical imaging method of claim 20, further comprising acquiring second energy signals associated with energy delivered during the surgical operation by one or more second energy delivery systems to the region of interest from above the bed top surface.

22. The real-time surgical imaging method of claim 20, further comprising:
reconstructing one or more second images of the region of interest based on the energy signals;
co-registering the one or more second images with one or more corresponding one or more pre-operative images; and
displaying co-registered images.
